(19) United States
(12) Patent Application Publication
(10) Pub. No.: US 2014/0248360 A1
(43) Pub. Date: Sep. 4, 2014

(54) NANOPARTICLE TUMOUR VACCINES

(71) Applicants: MIDATECH LIMITED, Oxford, Oxfordshire (GB); IMMUNOTOPE, INC., Doylestown, PA (US)

(72) Inventors: Thomas Rademacher, Oxford (GB); Ramila Philip, Ivyland, PA (US)

(21) Appl. No.: 14/343,435

(22) PCT Filed: Sep. 7, 2012
(86) PCT No.: PCT/EP2012/067579

§ 371 Date: May 14, 2014

Related U.S. Application Data
(60) Provisional application No. 61/531,730, filed on Sep. 7, 2011.

(51) Int. Cl. A61K 9/14; A61K 39/00

(52) U.S. Cl. CPC A61K 9/141; A61K 39/0011
USPC 424/489; 424/277.1; 435/375; 435/373

(57) ABSTRACT

The present invention provides a vaccine for the prophylactic or therapeutic treatment of a tumour in a mammalian subject, as well as methods of using the vaccine, including in treatment of tumours and in generating a CTL response. The vaccine comprises a plurality of nanoparticles and a pharmaceutically acceptable carrier, salt or diluents. The nanoparticles comprise a core comprising a metal and/or a semiconductor atom; and a corona comprising a plurality of ligands covalently linked to the core, wherein at least a first ligand of said plurality comprises a carbohydrate moiety that is covalently linked to the core via a first linker, and wherein at least a second ligand of said plurality comprises an epitopic peptide that is covalently linked to the core via a second linker, said second linker comprising a peptide portion and a non-peptide portion, wherein said peptide portion comprises the sequence X₁X₂Z₁, wherein X₁ is an amino acid selected from A and G; X₂ is an amino acid selected from A and G; and Z₁ is an amino acid selected from Y and F, and wherein said epitopic peptide forms at least a portion of or is derived from a Tumour-Associated Antigen (TAA).

### FIELD OF THE INVENTION

The present invention relates to substances and compositions useful in peptide-based vaccine strategies, in particular nanoparticle-mediated delivery of peptides in order to stimulate a T cell response. Vaccine strategies are directed to therapeutic and prophylactic treatment of tumours, such as lung cancer tumours.

### BACKGROUND TO THE INVENTION

Cytotoxic T lymphocytes (CTLs) are specialized T cells that function primarily by recognizing and killing cancerous cells or infected cells, but also by secreting soluble molecules referred to as cytokines that can mediate a variety of effects on the immune system. Evidence suggests that immunotherapy designed to stimulate a tumour-specific CTL response would be effective in controlling cancer. For example, it has been shown that human CTLs recognize sarcomas (Slovin, S. F. et al., J. Immunol., 137:3042-3048, (1987)), renal cell carcinomas (Schendel, D. J. et al., J. Immunol., 151:4209-4220, (1993)), colorectal carcinomas (Jacob, L. et al., Int. J. Cancer, 71:325-332, (1997)), ovarian carcinomas (Ioannides, C. G. et al., J. Immunol., 146:1700-1707, (1991)) (Peoples, G. E. et al., Surgery, 114:227-234, (1993)), pancreatic carcinomas (Peiper, M. et al., Eur. J. Immunol., 27:1115-1123, (1997); Wolfel, T. et al., Int. J. Cancer, 54:636-644, (1993)), squamous tumors of the head and neck (Yasumura, S. et al., Cancer Res., 53:1461-1468, (1993)), and squamous carcinomas of the lung (Slingluff, C. L. Jr et al., Cancer Res., 54:2731-2737, (1994); Yoshino, I. et al., Cancer Res., 54:3387-3390, (1994)). The largest number of reports of human tumor-reactive CTLs have concerned cancers (Boon, T. et al., Ann. Rev. Immunol., 12:337-365, (1994)). The ability of tumor-specific CTLs to mediate tumor regression, in both human (Rosenberg, S. A. et al., N. Engl. J. Med., 319:1676-1680, (1988)) and animal models (Celluzzi, C. M. et al., J. Exp. Med., 183:283-287, (1996); Mayordomo, J. I. et al., Nat. Med., 1:1297-1302, (1995); Zitvogel, L. et al., J. Exp. Med., 183:87-97, (1996)), suggests that methods directed at increasing CTL activity would likely have a beneficial effect with respect to tumour treatment.

In order for CTLs to kill or secrete cytokines in response to a cancer cell, the CTL must first recognize that cell as being cancerous. This process involves the interaction of the T cell receptor, located on the surface of the CTL, with what is generically referred to as an MHC-peptide complex which is located on the surface of the cancerous cell. MHC (Major Histocompatibility Complex)-encoded molecules have been subdivided into two types, and are referred to as class I and class II MHC-encoded molecules. In the human immune system, MHC molecules are referred to as human 30 leukocyte antigens (HLA). Within the MHC, located on chromosome six, are three different genetic loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, and HLA-B8 are examples of different class I MHC molecules that can be expressed from these loci. The present disclosure involves peptides that are associated with the HLA-A1, HLAA2, or HLA-Al 1 molecules, HLA-A1 supertypes, HLA-A2 supertypes, and HLA-All supertypes. A supertype is a group of HLA molecules that present at least one shared epitope. The present disclosure involves peptides that are associated with HLA molecules, and with the genes and proteins from which these peptides are derived.

The peptides that associate with the MHC molecules can either be derived from proteins made within the cell, in which case they typically associate with class I MHC molecules (Rock, K. L. and Golde, U., Ann. Rev. Immunol., 17:739-779, (1999)) or they can be derived from proteins that are acquired from outside of the cell, in which case they typically associate with class II MHC molecules (Watts, C., Ann. Rev. Immunol., 15:821-850, (1997)). Peptides that evoke a cancer-specific CTL response most typically associate with class I MHC molecules. The peptides that associate with a class I MHC molecule are typically nine amino acids in length, but can vary from a minimum length of eight amino acids to a maximum of fourteen amino acids in length. A class I MHC molecule with its bound peptide, or a class II MHC molecule with its bound peptide, is referred to as an MHC-peptide complex.

The process by which intact proteins are degraded into peptides is referred to as antigen processing. Two major pathways of antigen processing occur within cells (Rock, K. L. and Golde, U., Ann. Rev. Immunol., 17:739-779, (1999); Watts, C., Ann. Rev. Immunol., 15:821-850, (1997)). One pathway, which is largely restricted to cells that are antigen presenting cells such as dendritic cells, macrophages, and B cells, degrades proteins that are typically phagocytosed or endocytosed into the cell. Peptides derived in this pathway typically bind to class II MHC molecules. A second pathway of antigen processing is present in essentially all cells of the body. This second pathway primarily degrades proteins that are made within the cells, and the peptides derived from this pathway primarily bind to class I MHC molecules. It is the peptides from this second pathway of antigen processing that are referred to herein. Antigen processing by this latter pathway involves polypeptide synthesis and proteolysis in the cytoplasm. The peptides produced are then transported into the endoplasmic reticulum of the cell, associate with newly synthesized class I MHC molecules, and the resulting MHC-peptide complexes are then transported to the cell surface. Peptides derived from membrane and secreted proteins may also associate with Class I MHC molecules. In some cases these peptides correspond to the signal sequence of the proteins that are cleaved from the protein by the signal peptidase. In other cases, it is thought that some fraction of the membrane and secreted proteins are transported from the endoplasmic reticulum into the cytoplasm where processing subsequently occurs.

Once bound to the class I MHC molecule and displayed on the surface of a cell, the peptides are recognized by antigen-specific receptors on CTLs. Mere expression of the class I MHC molecule itself is insufficient to trigger the CTL to kill the target cell if the antigenic peptide is not bound to the class I MHC molecule. Several methods have been developed to identify the peptides recognized by CTL, each method relying on the ability of a CTL to recognize and kill only those cells expressing the appropriate class I MHC molecule with the peptide bound to it (Rosenberg, S. A., Immunity, 10:281-287, (1999)). Such peptides can be derived from a non-self source, such as a pathogen (for example, following the infection of a cell by a bacterium or a virus) or from a self-derived protein within a cell, such as a cancerous cell. Examples of sources of self-derived proteins in cancerous cells have been reviewed (Gilboa, E., Immunity, 11:263-270, (1999); Rosenberg, S. A., Immunity, 10:281-287, (1999)) and include: (i) mutated genes; (ii) aberrantly expressed genes such as an alternative open reading frame or through an intron-exon boundary; (iii) normal genes that are selectively expressed in only the tumour and the testis; and (iv) normal differentiation genes that are expressed in the tumour and the normal cellular counterpart.

Oberg et al., 2011, European Journal of Cell Biology, Vol. 90, pp. 582-592, describes regulation of T cell activation by Toll-like receptor (TLR) ligands. McKee et al., 2005, Journal of Translational Medicine, Vol. 3, p. 35, reviews implications and therapeutic strategies relating to T cell avidity and tumour recognition.

WO 2011/025572 describes CTL-inducing immunogens for prevention, treatment and diagnosis of cancer.

A significant challenge for the design and development of peptide-based vaccine therapy for treatment of tumours is the delivery of the epitope-containing peptides via the antigen processing machinery such that the peptides are presented bound to a class I MHC molecule and thereby stimulate a CTL response. It is frequently the case that administration of one or more adjuvants is necessary in order to induce an effective immune response. A number of adjuvants are considered too toxic for, e.g., human use.

Although many products have been developed for the treatment of cancer there is still a high demand for substances which have improved characteristics compared to the already known substances. In particular, in the field of vaccination, there is a need to provide products that are highly immunogenic, easily reproducible and highly effective, but do not cause severe side effects.

WO 2006/037979 describes nanoparticles comprising antigens and adjuvants, and immunogenic structures.

### BRIEF DESCRIPTION OF THE INVENTION

The present inventors have found that peptides bound to nanoparticles via certain linkers exhibit the ability to be internalised and processed by antigen presenting cells (APCs) such that the peptides are bound to MHC and induce a CTL response. In particular, tumour antigen associated (TAA) peptides delivered via nanoparticles are able to stimulate a high avidity tumour-specific CTL response even in the absence of adjuvants.

Accordingly, in a first aspect the present invention provides a vaccine for the prophylactic or therapeutic treatment of a tumour in a mammalian subject, said vaccine comprising a plurality of nanoparticles and a pharmaceutically acceptable carrier, salt or diluent, at least one of said nanoparticles comprising:

(i) a core comprising a metal and/or a semiconductor atom;

(ii) a corona comprising a plurality of ligands covalently linked to the core, wherein at least a first ligand of said plurality comprises a carbohydrate moiety that is covalently linked to the core via a first linker or wherein said first ligand of said plurality comprises glutathione, and wherein at least a second ligand of said plurality comprises an epitopic peptide that is covalently linked to the core via a second linker, said second linker comprising:
a peptide portion and a non-peptide portion, wherein said peptide portion comprises the sequence X₁X₂Z₁, wherein:
X₁ is an amino acid selected from A and G;
X₂ is an amino acid selected from A and G; and
Z₁ is an amino acid selected from Y and F,
   and wherein said epitopic peptide forms at least a portion of or is derived from a Tumour-Associated Antigen (TAA).

In some cases in accordance with the present invention the non-peptide portion of the second linker comprises C2-C15 alkyl and/or C2-C15 glycol, for example a thioethyl group or a thiopropyl group.

In some cases in accordance with the present invention the first ligand and/or said second ligand are covalently linked to the core via a sulphur-containing group, an amino-containing group, a phosphate-containing group or an oxygen-containing group.

The peptide portion of said second linker may comprise or consist of an amino acid sequence selected from:(i) AAY; and (ii) FLAAY (SEQ ID NO: 91). In certain cases, the second linker is selected from the group consisting of:
(i) HS—(CH₂)₂—CCNH-AAY;
(ii) HS—(CH₂)₂—CCNH-FLAAY;
(iii) HS—(CH₂)₃—CCNH-AAY;
(iv) HS—(CH₂)₃—CCNH-FLAAY;
(v) HS—(CH₂)₁₀—(CH₂OCH₂)₇—CCNH-AAY; and
(vi) HS—(CH₂)₁₀—(CH₂OCH₂)₇—CONH-FLAAY,
   wherein said second linker is covalently linked to said core via the thiol group of the non-peptide portion of the linker.

Preferably, the epitopic peptide is linked via its N-terminus to said peptide portion of said second linker. Thus, the second ligand may be selected from the group consisting of:
(i) HS—(CH₂)₂—CCNH-AAYZ₂;
(ii) HS—(CH₂)₂—CCNH-FLAAYZ₂;
(iii) HS—(CH₂)₃—CCNH-AAYZ₂;
(iv) HS—(CH₂)₃—CCNH-FLAAYZ₂;
(v) HS—(CH₂)₁₀—(CH₂OCH₂)₇—CONH-AAYZ₂; and
(vi) HS—(CH₂)₁₀—(CH₂OCH₂)₇—CONH-FLAAYZ₂ᵣ
   wherein Z₂ represents said epitopic peptide.

Preferably, the epitopic peptide binds to a class I Major Histocompatibility Complex (MHC) molecule or is capable of being processed so as to bind to a class I MHC molecule.

The epitopic peptide may consists of a sequence of 8 to 40 amino acid residues, such as a sequence of 8 to 12 amino acid residues. The epitopic peptide may be capable of being presented by a class I MHC molecule so as to stimulate a Cytotoxic T Lymphocyte (CTL) response.

In some cases in accordance with the present invention the TAA is a lung cancer antigen. Said lung cancer may be selected from: small-cell lung carcinoma, non-small-cell lung carcinoma and adenocarcinoma.

The epitopic peptide may in some cases comprise or consists of an amino acid sequence selected from SEQ ID NOS: 1 to 86. These epitopic peptides are described in detail in WO 2011/025572, the entire contents of which is expressly incorporated herein by reference. In particular, the epitopic peptide may comprise or consist of an amino acid sequence selected from the group consisting of:

(SEQ ID NO: 82)
VLVPVLVMV;
(SEQ ID NO: 29)
KIYQWINEL;
(SEQ ID NO: 33)
KLGEFAKVLEL;
(SEQ ID NO: 19)
GMYGKIAVMEL;
(SEQ ID NO: 34)
KLIPFLEKL;
and
(SEQ ID NO: 67)
RLLEVPVML.

In some cases in accordance with the present invention, the carbohydrate moiety of said first ligand comprises a monosaccharide and/or a disaccharide. In particular, said carbohydrate moiety may comprise glucose, mannose, fucose and/or N-acetylglucosamine.

In some cases in accordance with the present invention, said plurality of ligands comprises one or more ligands selected from the group consisting of: glucose, N-acetylglucosamine and glutathione, in addition to the one or more ligands comprising said epitopic peptides.

In some cases in accordance with the present invention, said plurality of ligands comprises:
(a) glucose;
(b) N-acetylglucosamine;
(c) glutathione;
(d) glucose and N-acetylglucosamine;
(e) glucose and glutathione;
(f) N-acetylglucosamine and glutathionie; or
(g) glucose, N-acetylglucosamine and glutathione,
   in addition to said ligand comprising an epitopic peptide.

In some cases in accordance with the present invention, said first linker comprises C2-C15 alkyl and/or C2-C15 glycol. In particular, said first ligand may comprise 2′-thioethyl-β-D-glucopyranoside or 2′-thioethyl-α-D-glucopyranoside covalently attached to the core via the thiol sulphur atom.

In some cases in accordance with the present invention, the nanoparticle comprises at least 10, at least 20, at least 30, at least 40 or at least 50 carbohydrate-containing ligands and/or glutathione ligands.

In some cases in accordance with the present invention, the nanoparticle comprises at least 1, at least 2, at least 3, at least 4 or at least 5 epitopic peptide-containing ligands.

In some cases in accordance with the present invention, the molar ratio of carbohydrate-containing ligands and/or glutathione ligands to epitopic peptide-containing ligands is in the range 5:1 to 100:1, such as in the range 10:1 to 30:1.

The diameter of the core of the nanoparticle may be in the range 1 nm to 5 nm. The diameter of the nanoparticle including its ligands may be in the range 5 nm to 20 nm, optionally 5 nm to 15 nm or 8 nm to 10 nm.

In some cases in accordance with the present invention, the at least one nanoparticle comprises at least two epitopic peptide-containing ligands, and wherein the epitopic peptide of each of the at least two epitopic peptide-containing ligands differ. The epitopic peptides of said at least two epitopic peptide-containing ligands may each form at least a portion of or may each be derived from a different lung cancer TAA.

In some cases in accordance with the present invention, the vaccine comprises a first species of said nanoparticle having a first epitopic peptide-containing ligand and a second species of said nanoparticle having a second epitopic peptide-containing ligand, wherein the epitopic peptides of said first and second species differ. In particular, the epitopic peptides of each of said first and second species of nanoparticle may each form at least a portion of or may each be derived from a different lung cancer TAA.

In some cases in accordance with the present invention, the vaccine may comprise a pool of at least 3, at least 4, at least 5 or at least 10 different species of nanoparticle, each species having a different epitopic peptide.

In some cases in accordance with the present invention, the vaccine may further comprise at least one adjuvant. The adjuvant may be covalently attached to the core of at least one nanoparticle. The adjuvant may comprise (S)-(2,3-bis(palmitoyloxy)-(2RS)-propyl)-N-palmitoyl-(R)-Cys-(S)-Ser(S)-Lys₄-OH (“Pam₃Cys”).

In some cases in accordance with the present invention, the vaccine is substantially free of adjuvant or wherein the only adjuvant effect is provided by the nanoparticles.

In a further aspect, the present invention provides a vaccine as defined in accordance with the first aspect of the invention for use in medicine. The vaccine may be for use in a prophylactic or therapeutic method of treatment of a cancer in a mammalian subject (e.g. human subject), such as lung cancer.

In a further aspect, the present invention provides use of a vaccine as defined in any one of the preceding claims in the preparation of a medicament for the prophylactic or therapeutic treatment of a cancer in a mammalian subject, such as lung cancer.

The vaccine of the invention may be for administration via lymphatic uptake.

In a further aspect, the present invention provides a method of prophylactic or therapeutic treatment of a cancer (e.g. lung cancer), comprising administering a prophylactically or therapeutically sufficient amount of a vaccine in accordance with the first aspect of the invention to a mammalian subject in need thereof.

In a further aspect, the present invention provides an in vitro or in vivo method for generating a Cytotoxic T Lymphocyte (CTL) response, comprising:
(i) contacting at least one antigen presenting cell (APC) with a vaccine as defined in accordance with the first aspect of the invention, such that said epitopic peptide is presented on a class I MHC molecule of said APC; and
(ii) contacting said at least one APC of (i) with at least one CTL cell, such that said CTL cell is activated by said APC to generate a CTL response that is specific for said epitopic peptide.

The APC may be cultured in the presence of said vaccine, and, simultaneously or sequentially, co-cultured with said CTL cell. In some cases, the APC may be subjected to a washing step after being contacted with the vaccine before being co-cultured with said CTL cell. The method may further comprise administering the CTL cell to a mammalian subject.

In some cases in accordance with the method of this aspect of the invention, said at least one CTL cell exhibits higher avidity for an MHC-peptide complex that comprises said epitopic peptide displayed on a class I MHC molecule, wherein said higher avidity is higher compared with the avidity for said MHC-peptide complex exhibited by a CTL cell activated by an APC that has been contacted with the same epitopic peptide in free peptide form not linked to a nanoparticle.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows SIINFEKL (SEQ ID NO: 87) presentation from GNP: LKb cells were seeded into 24-well plates and allowed to adhere overnight. Next, GNPs were pulsed to the equivalent of 1 ug/mL peptide (Green), 0.1 ug/mL (Blue), or 0.01 ug/mL (Red). After two hours, cells were washed, and subjected to (A-D) flow cytometric labelling with 25.D1.16 (Angel) antibody, or (E) combined with B3Z CTL for overnight co-culture. The next day, cells were lysed and Beta-galactosidase activity was measured.

FIG. 2: GNP presentation compared to free peptide presentation: (A-B) GNPs 8 and 9 were separated by Sephadex column into 15 fractions, which were then analyzed by UV absorption for protein levels. The red line (indicated by circles) in FIG. 2A represents free peptide alone. (C—H) LKb cells were pulsed for 2 hrs with noted GNPs or corresponding free peptide at the noted concentration (1 ug/mL peptide (Green), 0.1 ug/mL (Blue), or 0.01 ug/mL (Red)). Readouts are by (C-F) flow cytometry or (G-H) B3Z assay. (I-N) LKb were pulsed with the noted free peptide for 2 hrs on ice (I-K) or at 37° C. (L-N). Next, cells were analyzed by flow cytometry for presentation of SIINFEKL (SEQ ID NO: 87). Red portions of the histogram represent positive staining as compared to unpulsed cells.

FIG. 3: GNP presentation from preparations lacking free peptide: (A-B) New preparations of GNPs 8 and 9 were separated by Sephadex G-50 column into 15 fractions, which were then analyzed by UV absorption for protein levels. Arrows indicate which fractions were pooled for further use. (C-E,F) LKb cells were pulsed for 2 hrs with noted previous preparations of GNPs (old GNP) or newer preparations from (A and B) (new GNP) at the noted concentrations (1 ug/mL peptide (Green), or 0.1 ug/mL (Red)). Readouts are by (C-E) flow cytometry or (F) B3Z assay.

FIG. 4: shows CTL response measured by number of IFN-gamma producing cells per million splenocytes for HLA transgenic mice immunized with GNPs with individual lung cancer antigens.

FIG. 5: shows CTL response measured by number of IFN-gamma producing cells per million splenocytes for HLA transgenic mice immunized with pooled GNPs with three different lung cancer antigens.

FIG. 6: shows number of IFN-gamma producing cells per 10⁵ human PBMCs following stimulation with pooled GNPs with six different lung cancer antigens as compared with pooled free peptide controls.

FIG. 7: (A-B) shows IFN-gamma ELISpot assay results in which lung antigens (KIY, KLG, GMY) were tested in NPs with Glc, GlcNAc, GSH corona for activation of CTL in vivo in a HLA-A2 transgenic mouse model. Pooled 3 lung peptides (KIY, KLG, GMY) in NPs with Glc, GlcNAc, GSH corona or free pooled peptides+montanide were used to immunize mice. After 3 immunizations, splenocytes from immunized mice were mixed with various target cells (T2—empty HLA-A2+ cells, N lung—HLA-A2+ normal lung, HLA-A2+ Lung tumor cells—H522, 5865, 5944) to measure IFN-gamma secretion in an ELISpot assay.

FIG. 8: (A-D) shows antigen specific CTL degranulation marker CD107a analysis in the splenocytes in response to peptide loaded T2 cells and well as lung tumor cells for (A) glucose NPs, (B) GlcNAc NPs, (C) GSH NPs, and (D) free peptides+adjuvant.

### DETAILED DESCRIPTION OF THE INVENTION

In describing the present invention, the following terms will be employed, and are intended to be defined as indicated below.

As used herein, “nanoparticle” refers to a particle having a nanomeric scale, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry.

Nanoparticles comprising a plurality of carbohydrate-containing ligands have been described in, for example, WO 2002/032404, WO 2004/108165, WO 2005/116226, WO 2006/037979, WO 2007/015105, WO 2007/122388, WO 2005/091704 (the entire contents of each of which is expressly incorporated herein by reference) and such nanoparticles may find use in accordance with the present invention. Moreover, gold-coated nanoparticles comprising a magnetic core of iron oxide ferrites (having the formula XFe₂O₄, where X=Fe, Mn or Co) functionalised with organic compounds (e.g. via a thiol-gold bond) are described in EP2305310 (the entire contents of which is expressly incorporated herein by reference) and are specifically contemplated for use as nanoparticles/nanoparticle cores in accordance with the present invention.

As used herein, “corona” refers to a layer or coating, which may partially or completely cover the exposed surface of the nanoparticle core. The corona includes a plurality of ligands which include at least one carbohydrate moiety, one surfactant moiety and/or one glutathione moiety. Thus, the corona may be considered to be an organic layer that surrounds or partially surrounds the metallic core. In certain embodiments the corona provides and/or participates in passivating the core of the nanoparticle. Thus, in certain cases the corona may include a sufficiently complete coating layer substantially to stabilise the metal-containing core. However, it is specifically contemplated herein that certain nanoparticles having cores, e.g., that include a metal oxide-containing inner core coated with a noble metal may include a corona that only partially coats the core surface. In certain cases the corona facilitates solubility, such as water solubility, of the nanoparticles of the present invention.

### Nanoparticles

Nanoparticles are small particles, e.g. clusters of metal or semiconductor atoms, that can be used as a substrate for immobilising ligands.

Preferably, the nanoparticles have cores having mean diameters between 0.5 and 50 nm, more preferably between 0.5 and 10 nm, more preferably between 0.5 and 5 nm, more preferably between 0.5 and 3 nm and still more preferably between 0.5 and 2.5 nm. When the ligands are considered in addition to the cores, preferably the overall mean diameter of the particles is between 5.0 and 100 nm, more preferably between 5 and 50 nm and most preferably between 5 and 10 nm. The mean diameter can be measured using techniques well known in the art such as transmission electron microscopy.

The core material can be a metal or semiconductor and may be formed of more than one type of atom. Preferably, the core material is a metal selected from Au, Fe or Cu. Nanoparticle cores may also be formed from alloys including Au/Fe, Au/Cu, Au/Gd, Au/Fe/Cu, Au/Fe/Gd and Au/Fe/Cu/Gd, and may be used in the present invention. Preferred core materials are Au and Fe, with the most preferred material being Au. The cores of the nanoparticles preferably comprise between about 100 and 500 atoms (e.g. gold atoms) to provide core diameters in the nanometre range. Other particularly useful core materials are doped with one or more atoms that are NMR active, allowing the nanoparticles to be detected using NMR, both in vitro and in vivo. Examples of NMR active atoms include Mn⁺², Gd⁺³, Eu⁺², Cu⁺², V⁺², Co⁺², Ni⁺², Fe⁺², Fe⁺³ and lanthanides⁺³, or the quantum dots described elsewhere in this application.

Nanoparticle cores comprising semiconductor atoms can be detected as nanometre scale semiconductor crystals are capable of acting as quantum dots, that is they can absorb light thereby exciting electrons in the materials to higher energy levels, subsequently releasing photons of light at frequencies characteristic of the material. An example of a semiconductor core material is cadmium selenide, cadmium sulphide, cadmium tellurium. Also included are the zinc compounds such as zinc sulphide.

In some embodiments, the core of the nanoparticles may be magnetic and comprise magnetic metal atoms, optionally in combination with passive metal atoms. By way of example, the passive metal may be gold, platinum, silver or copper, and the magnetic metal may be iron or gadolinium. In preferred embodiments, the passive metal is gold and the magnetic metal is iron. In this case, conveniently the ratio of passive metal atoms to magnetic metal atoms in the core is between about 5:0.1 and about 2:5. More preferably, the ratio is between about 5:0.1 and about 5:1. As used herein, the term “passive metals” refers to metals which do not show magnetic properties and are chemically stable to oxidation.

The passive metals may be diamagnetic or superparamagnetic. Preferably, such nanoparticles are superparamagnetic.

Examples of nanoparticles which have cores comprising a paramagnetic metal, include those comprising Mn⁺², Gd⁺³, Eu⁺², Cu⁺², V⁺², Co⁺², Ni⁺², Fe⁺², Fe⁺³ and lanthanides⁺³.

Other magnetic nanoparticles may be formed from materials such as MnFe (spinel ferrite) or CoFe (cobalt ferrite) can be formed into nanoparticles (magnetic fluid, with or without the addition of a further core material as defined above. Examples of the self-assembly attachment chemistry for producing such nanoparticles is given in Biotechnol. Prog., 19:1095-100 (2003), J. Am. Chem. Soc. 125:9828-33 (2003), J. Colloid Interface Sci. 255:293-8 (2002).

In some embodiments, the nanoparticle or its ligand comprises a detectable label. The label may be an element of the core of the nanoparticle or the ligand. The label may be detectable because of an intrinsic property of that element of the nanoparticle or by being linked, conjugated or associated with a further moiety that is detectable. Preferred examples of labels include a label which is a fluorescent group, a radionuclide, a magnetic label or a dye. Fluorescent groups include fluorescein, rhodamine or tetramethyl rhodamine, Texas-Red, Cy3, Cy5, etc., and may be detected by excitation of the fluorescent label and detection of the emitted light using Raman scattering spectroscopy (Y. C. Cao, R. Jin, C. A. Mirkin, Science 2002, 297: 1536-1539).

In some embodiments, the nanoparticles may comprise a radionuclide for use in detecting the nanoparticle using the radioactivity emitted by the radionuclide, e.g. by using PET, SPECT, or for therapy, i.e. for killing target cells. Examples of radionuclides commonly used in the art that could be readily adapted for use in the present invention include ^{99m}Tc, which exists in a variety of oxidation states although the most stable is TcO⁴⁻; ³²P or ³³P; ⁵⁷Co; ⁵⁹Fe; ⁶⁷Cu which is often used as Cu²⁺ salts; ⁶⁷Ga which is commonly used a Ga³⁺ salt, e.g. gallium citrate; ⁶⁸Ge; ⁸²Sr; ⁹⁹Mo; ¹⁰³Pd; ¹¹¹In which is generally used as In³⁺ salts; ¹²⁵I or ¹³¹I which is generally used as sodium iodide; ¹³⁷Cs; ¹⁵³Gd; ¹⁵³Sm; ¹⁵⁸Au; ¹⁸⁶Re; ²⁰¹Tl generally used as a Tl⁺ salt such as thallium chloride; ³⁹Y³⁺; ⁷¹Lu³⁺; and ²⁴Cr²⁺. The general use of radionuclides as labels and tracers is well known in the art and could readily be adapted by the skilled person for use in the aspects of the present invention. The radionuclides may be employed most easily by doping the cores of the nanoparticles or including them as labels present as part of ligands immobilised on the nanoparticles.

Additionally or alternatively, the nanoparticles of the present invention, or the results of their interactions with other species, can be detected using a number of techniques well known in the art using a label associated with the nanoparticle as indicated above or by employing a property of them. These methods of detecting nanoparticles can range from detecting the aggregation that results when the nanoparticles bind to another species, e.g. by simple visual inspection or by using light scattering (transmittance of a solution containing the nanoparticles), to using sophisticated techniques such as transmission electron microscopy (TEM) or atomic force microscopy (AFM) to visualise the nanoparticles. A further method of detecting metal particles is to employ plasmon resonance that is the excitation of electrons at the surface of a metal, usually caused by optical radiation. The phenomenon of surface plasmon resonance (SPR) exists at the interface of a metal (such as Ag or Au) and a dielectric material such as air or water. As changes in SPR occur as analytes bind to the ligand immobilised on the surface of a nanoparticle changing the refractive index of the interface. A further advantage of SPR is that it can be used to monitor real time interactions. As mentioned above, if the nanoparticles include or are doped with atoms which are NMR active, then this technique can be used to detect the particles, both in vitro or in vivo, using techniques well known in the art. Nanoparticles can also be detected using a system based on quantitative signal amplification using the nanoparticle-promoted reduction of silver (I). Fluorescence spectroscopy can be used if the nanoparticles include ligands as fluorescent probes. Also, isotopic labelling of the carbohydrate can be used to facilitate their detection.

### Administration and Treatment

The nanoparticle-containing vaccine compositions of the invention may be administered to patients by any number of different routes, including enteral or parenteral routes. Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes.

Administration be performed e.g. by injection, or ballistically using a delivery gun to accelerate their transdermal passage through the outer layer of the epidermis. The nanoparticles can then be taken up, e.g. by dendritic cells, which mature as they migrate through the lymphatic system, resulting in modulation of the immune response and vaccination against the epitopic peptide and/or the antigen from which the epitopic peptide was derived or of which it forms a part. The nanoparticles may also be delivered in aerosols. This is made possible by the small size of the nanoparticles.

The exceptionally small size of the nanoparticles of the present invention is a great advantage for delivery to cells and tissues, as they can be taken up by cells even when linked to targeting or therapeutic molecules. Thus, the nanoparticles may be internalised by APCs, the epitopic peptides processed and presented via class I MHC.

The nanoparticles of the invention may be formulated as pharmaceutical compositions that may be in the forms of solid or liquid compositions. Such compositions will generally comprise a carrier of some sort, for example a solid carrier such as gelatine or an adjuvant or an inert diluent, or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parenterally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual.

The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, New York, USA); Remington's Pharmaceutical Sciences, 20th Edition, 2000, pub. Lippincott, Williams & Wilkins; and Handbook of Pharmaceutical Excipients, 2nd edition, 1994. By way of example, and the compositions are preferably administered to patients in dosages of between about 0.01 and 100 mg of active compound per kg of body weight, and more preferably between about 0.5 and 10 mg/kg of body weight.

It will be understood that where treatment of tumours is concerned, treatment includes any measure taken by the physician to alleviate the effect of the tumour on a patient. Thus, although complete remission of the tumour is a desirable goal, effective treatment will also include any measures capable of achieving partial remission of the tumour as well as a slowing down in the rate of growth of a tumour including metastases. Such measures can be effective in prolonging and/or enhancing the quality of life and relieving the symptoms of the disease.

### Immunotherapy

The compositions of the invention, such as the vaccines as defined in the claims, may be used for the prophylaxis and treatment of diseases such as cancer, and more particularly for immunotherapy.

In the present invention, the term “vaccination” means an active immunization, that is an induction of a specific immune response due to administration, e.g. via the subcutaneous, intradermal, intramuscular, oral or nasal routes, of small amounts of an antigen which is recognized by the vaccinated individual as foreign and is therefore immunogenic in a suitable formulation. The antigen is thus used as a “trigger” for the immune system in order to build up a specific immune response against the antigen.

In accordance with the present invention, vaccination may be therapeutic or prophylactic. By way of example, it might be possible to achieve a prophylactic protection against the breakout of a cancer disease by vaccination of individuals who do not suffer from cancer. Examples of individuals for whom such a prophylactic vaccination might be applied are individuals who have an increased risk of developing a cancer disease, although this application is not limited to such individuals. Patients being at risk of cancer can already have developed tumours, either as primary tumours or metastases, or show predisposition for cancer.

For the active immunization of cancer patients according to the invention, the nanoparticles are typically formulated as vaccines. Preferably, such pharmaceutical preparations contain a pharmaceutically acceptable carrier which, by way of example, may further comprise auxiliary substances, buffers, salts and/or preserving agents. The pharmaceutical preparations may, e.g., be used for the prophylaxis and therapy of cancer-associated conditions, such as metastasis formation, in cancer patients. In doing so, antigen-presenting cells are specifically modulated in vivo or also ex vivo so as to generate the immune response against the TAAs.

For the active immunization with the specific antigens or the antigen combination usually a vaccine formulation is used which contains the immunogen—be it a natural TAA or its epitope, mimic or neoepitope mimic,—mostly at low concentrations, e.g. in an immunogenic amount ranging from 0.01 μg to 10 mg, yet the dosage range can be increased up a range of 100 to 500 mg. Depending on the immunogenicity of the vaccination antigen which is, e.g., determined by sequences of a foreign species or by derivatization, or also depending on the auxiliary substances or adjuvants, respectively, used, the suitable immunogenic dose can be chosen e.g. in the range of from 0.01 μg to 1 mg, preferably 100 μg to 500 μg. A depot vaccine which is to be delivered to the organism over an extended period of time may, however, also contain much higher amounts of vaccination antigen, e.g. at least 1 mg to more than 100 mg.

The concentration will depend on the amount of liquid or suspended vaccine administered. A vaccine usually is provided in ready-to-use syringes or ampoules having a volume ranging from 0.01 to 1 ml, preferably 0.1 to 0.75 ml.

The vaccination antigen of a component of vaccine preferably is presented in a pharmaceutically acceptable carrier which is suitable for subcutaneous, intramuscular and also intradermal or transdermal administration. A further mode of administration functions via the mucosal pathway, e.g. vaccination by nasal or peroral administration. If solid substances are employed as auxiliary agent for the vaccine formulation, e.g. an adsorbate, or a suspended mixture, respectively, of the vaccine antigen with the auxiliary agent will be administered. In special embodiments, the vaccine is presented as a solution or a liquid vaccine in an aqueous solvent.

Preferably, vaccination units of a tumour vaccine are already provided in a suitable ready-to-use syringe or ampoule. A stable formulation of the vaccine may advantageously be put on the market in a ready to use form. Although a content of preserving agents, such as thimerosal or other preserving agents with an improved tolerability, is not necessarily required, yet it may be provided in the formulation for a longer stability at storage temperatures of from refrigerating temperatures up to room temperature. The vaccine according to the invention may, however, also be provided in frozen or lyophilized form and may be thawed or reconstituted, respectively, upon demand.

In certain cases in accordance with the present invention the immunogenicity of the vaccine of the invention may be increased by by employing adjuvants. For this purpose, solid substances or liquid vaccine adjuvants are used, e.g. aluminum hydroxide (Alu-Gel) or aluminum phosphate, growth factors, lymphokines, cytokines, such as IL-2, IL-12, GM-CSF, gamma interferon, or complement factors, such as C3d, further liposome preparations, or also formulations with additional antigens against which the immune system has already generated a strong immune response, such as tetanus toxoid, bacterial toxins, such as Pseudomonas exotoxins, and derivatives of lipid A and lipopolysaccharide.

In certain cases, no additional adjuvant is employed, in particular, the examples described herein show that efficient generation of a CTL response is achieved using nanoparticles of the invention without any added adjuvant (c.f. free peptides which are generally administered with one or more adjuvants). This is highly beneficial in certain circumstances as a number of adjuvants are considered to be toxic or otherwise unsuitable for human use.

### Epitopic Peptides

In certain preferred cases in accordance with the present invention, the epitopic peptide may comprise or consist of an amino acid sequence set forth in Table 1 below.

| SEQ | SWISSPROT | | |
|---|---|---|---|
| ID | IDENTIFICATION | ACTIVE | |
| NO | NUMBER | FRAGMENT | PARENT PROTEIN NAME |
| SEQ | Q7Z460 | AEYDNFFQHL | CLIP-associating protein 1-Homo |
| ID | | | sapiens (Human)-[CLAP1_HUMAN] |
| NO: 1 | | | |
| SEQ | P61923 | ALILEPSLYTV | Coatomer subunit zeta-1-Homo |
| ID | | | sapiens (Human)-[COPZ1_HUMAN] |
| NO: 2 | | | |
| SEQ | Q13075 | ALLGLDAVQL | Baculoviral IAP repeat-containing |
| ID | | | protein 1-Homo sapiens (Human)- |
| NO: 3 | | | [BIRC1_HUMAN] |
| SEQ | Q6UXD5 | DDVPERGLI | Seizure 6-like protein 2 precursor- |
| ID | | | Homo sapiens (Human)-[SE6L2_HUMAN] |
| NO: 4 | | | |
| SEQ | P05109 | DINTDGAVNF | Protein S100-A8-Homo sapiens |
| ID | | | (Human)-[S10A8_HUMAN] |
| NO: 5 | | | |
| SEQ | Q5TZA2 | DLDPEAVRGAL | Rootletin-Homo sapiens (Human)- |
| ID | | | [CROCC_HUMAN |
| NO: 6 | | | |
| SEQ | Q9UGL1 | DPFAFIHKI | Lysine-specific demethylase 5B-Homo |
| ID | | | sapiens (Human)-[JAD1B_HUMAN] |
| NO: 7 | | | |
| SEQ | Q6IA86 | DQFQKVLSL | ELP2 HUMAN Elongator complex protein |
| ID | | | 2 (ELP2) (STAT3-interacting protein) |
| NO: 8 | | | (StIP1) (SHINC-2) |
| SEQ | P79381 | DSPVGLAAYIL | HYEP_PIG RecName: Full = Epoxide |
| ID | | | hydrolase 1; AltName: Full = Microsomal |
| NO: 9 | | | epoxide hydrolase; AltName: |
| | | | Full = Epoxide hydratase |
| SEQ | O14525 | DVIVKTPCPVV | Astrotactin-1 precursor-Homo |
| ID | | | sapiens (Human)-[ASTN1_HUMAN] |
| NO: 10 | | | |
| SEQ | Q6NUJ1 | EAVRSNLTL | Proactivator polypeptide-like 1 |
| ID | | | precursor [Contains: Saposin A-like; |
| NO: 11 | | | Saposin B-Val-like; Saposin B-like; |
| | | | Saposin C-like; Saposin D-like]- |
| | | | Homo sapiens (Human)-[SAPL1_HUMAN] |
| SEQ | Q86YQ2 | EINTVSIQVK | LATH HUMAN Latherin precursor (Breast |
| ID | | | cancer and salivary gland-expressed |
| NO: 12 | | | protein) |
| SEQ | Q9UHY8 | ELNEILEEI | Fasciculation and elongation protein |
| ID | | | zeta-2-Homo sapiens (Human)- |
| NO: 13 | | | [FEZ2_HUMAN] |
| SEQ | O75325 | EMLPNLEIL | Leucine-rich repeat neuronal protein |
| ID | | | 2 precursor-Homo sapiens (Human)- |
| NO: 14 | | | [LRRN2_HUMAN] |
| SEQ | Q09666 | EMNIKVPKI | Neuroblast differentiation-associated- |
| ID | | | protein AHNAK-Homo sapiens (Human)- |
| NO: 15 | | | [AHNK_HUMAN] |
| SEQ | P32004 | EVEEGESVVLPC | Neural cell adhesion molecule L1 |
| ID | | | precursor-Homo sapiens (Human)- |
| NO: 16 | | | [L1CAM_HUMAN] |
| SEQ | Q96K83 | EVVNDLNTL | Zinc finger protein 521-Homo |
| ID | | | sapiens (Human)-[ZN521_HUMAN] |
| NO: 17 | | | |
| SEQ | P54819 | FLLDGFPRTV | KAD2_HUMAN RecName: Full = Adenylate |
| ID | | | kinase isoenzyme 2, mitochondrial; |
| NO: 18 | | | Short = AK 2; AltName: Full = ATP-AMP |
| | | | transphosphorylase |
| | | | 2gi\|75062006\|sp\|Q5REI7.3\|KAD2_PONAB |
| | | | Adenylate kinase isoenzyme 2, |
| | | | mitochondrial (AK 2) (ATP-AMP |
| | | | transphosphorylase 2) |
| SEQ | Q16531 | GMYGKIAVMEL | DNA damage-binding protein 1-Homo |
| ID | | | sapiens (Human)-[DDB1_HUMAN] |
| NO: 19 | | | |
| SEQ | Q5TCZ1.1 | HYVYIINV | SPD2A_HUMAN RecName: Full = SH3 and PX |
| ID | | | domain-containing protein 2A; |
| NO: 20 | | | AltName: Full = SH3 multiple domains |
| | | | protein 1; AltName: Full = Five SH3 |
| | | | domain-containing protein; AltName: |
| | | | Full = Adaptor protein TKS5 |
| SEQ | A6H8Y1 | ILDVIDDTI | Transcription factor TFIIIB component |
| ID | | | B″ homolog-Homo sapiens (Human)- |
| NO: 21 | | | [BDP1_HUMAN] |
| SEQ | P10912 | ILTTSVPVYSL | Growth hormone receptor precursor- |
| ID | | | Homo sapiens (Human)-[GHR_HUMAN] |
| NO: 22 | | | |
| SEQ | Q0P6H9 | IMPVHLLML | Transmembrane protein 62-Homo |
| ID | | | sapiens (Human)-[TMM62_HUMAN] |
| NO: 23 | | | |
| SEQ | Q70CQ2 | KDNIPMLL | Ubiquitin carboxyl-terminal hydrolase |
| ID | | | 34-Homo sapiens (Human)- |
| NO: 24 | | | [UBP34_HUMAN] |
| SEQ | Q9NZJ7 | KIFKEEGLLGF | Mitochondrial carrier homolog 1- |
| ID | | | Homo sapiens (Human)-[MTCH1_HUMAN] |
| NO: 25 | | | |
| SEQ | P52385 | KIKTLLNEL | Probable processing and transport |
| ID | | | protein-Human herpesvirus 7 (strain |
| NO: 26 | | | JI) (HHV-7) (Human T lymphotropic |
| | | | virus)-[PRTP_HHV7J] |
| SEQ | Q9HBX8 | KILMLQNNQ | Leucine-rich repeat-containing G- |
| ID | | | protein coupled receptor 6 precursor- |
| NO: 27 | | | Homo sapiens (Human)-[LGR6_HUMAN] |
| SEQ | | | |
| ID | Q5BJE1 | KINELNEEL | Uncharacterized protein C18orf34- |
| NO: 28 | | | Homo sapiens (Human)-[CR034_HUMAN] |
| SEQ | Q92600 | KIYQWINEL | Cell differentiation protein RCD1 |
| ID | | | homolog-Homo sapiens (Human)- |
| NO: 29 | | | [RCD1_HUMAN] |
| SEQ | Q92947 | KLADMLTEITL | Glutaryl-CoA dehydrogenase, |
| ID | | | mitochondrial precursor-Homo |
| NO: 30 | | | sapiens (Human)-[GCDH HUMAN] |
| SEQ | Q9Y5Q8 | KLFDIRPIW | General transcription factor 3C |
| ID | | | polypeptide 5-Homo sapiens (Human)- |
| NO: 31 | | | [TF3C5_HUMAN] |
| SEQ | P51968 | KLFIGGLSF | Heterogeneous nuclear |
| ID | | | ribonucleoproteins A2/B1-Homo |
| NO: 32 | | | sapiens (Human)-[ROA2_HUMAN] |
| SEQ | P30153 | KLGEFAKVLEL | Serine/threonine-protein phosphatase |
| ID | | | 2A 65 kDa regulatory subunit A alpha |
| NO: 33 | | | isoform-Homo sapiens (Human)- |
| | | | [2AAA_HUMAN] |
| SEQ | Q05D32 | KLIPFLEKL | CTD small phosphatase-like protein 2- |
| ID | | | Homo sapiens (Human)- |
| NO: 34 | | | [CTSL2_HUMAN] |
| SEQ | P31327 | KLNEINEKI | Carbamoyl-phosphate synthase |
| ID | | | [ammonia], mitochondrial precursor- |
| NO: 35 | | | Homo sapiens (Human)-[CPSM_HUMAN] |
| SEQ | Q5R4P0 | KLSLVAAML | 78 kDa glucose-regulated protein |
| ID | | | precursor-Homo sapiens (Human)- |
| NO: 36 | | | [GRP78_HUMAN] |
| SEQ | P47756 | KLTSTVMLW | CAPZB_HUMAN F-actin-capping protein |
| ID | | | subunit beta (CapZ beta) |
| NO: 37 | | | |
| SEQ | Q5RBD7 | KLYEFVHSF | DJB15_PONAB DnaJ homolog subfamily B |
| ID | | | member 15 |
| NO: 38 | | | |
| SEQ | Q8N4J0 | KLYPWIHQF | CI041_HUMAN UPF0586 protein C9orf41 |
| ID | | | |
| NO: 39 | | | |
| SEQ | Q14204 | KLYQEMFAW | Cytoplasmic dynein 1 heavy chain 1- |
| ID | | | Homo sapiens (Human)-[DYHC1_HUMAN] |
| NO: 40 | | | |
| SEQ | Q7Z4H8 | KMFSDEILL | KDEL motif-containing protein 2 |
| ID | | | precursor-Homo sapiens (Human)- |
| NO: 41 | | | [KDEL2_HUMAN] |
| SEQ | Q99729 | KMFVGGLSW | Heterogeneous nuclear |
| ID | | | ribonucleoprotein A/B-Homo sapiens |
| NO: 42 | | | (Human)-[ROAA_HUMAN] |
| SEQ | Q9NY93 | KSLLFVNTL | DDX56_HUMAN RecName: Full = Probable |
| ID | | | ATP-dependent RNA helicase DDX56; |
| NO: 43 | | | AltName: Full = DEAD box protein 56; |
| | | | AltName: Full = ATP-dependent 61 kDa |
| | | | nucleolar RNA helicase; AltName: |
| | | | Full = DEAD-box protein 21 |
| SEQ | Q9H579 | LTIKSIITL | Uncharacterized protein C20orf132- |
| ID | | | Homo sapiens (Human)-[CT132_HUMAN] |
| NO: 44 | | | |
| SEQ | P51797 | LTLLNPRMIV | Chloride channel protein 6-Homo |
| ID | | | sapiens (Human)-[CLCN6_HUMAN] |
| NO: 45 | | | |
| SEQ | Q9Y467 | LVEELSLQEA | Sal-like protein 2-Homo sapiens |
| ID | | | (Human)-[SALL2_HUMAN] |
| NO: 46 | | | |
| SEQ | Q695T7 | LVFQTCDI | S6A19 HUMAN Sodium-dependent neutral |
| ID | | | amino acid transporter B(0) (System |
| NO: 47 | | | B(0) neutral amino acid transporter) |
| | | | (B(0)AT1) (Solute carrier family 6 |
| | | | member 19) |
| SEQ | Q8NDA8 | LVMSNQKEVL | HEAT repeat-containing protein |
| ID | | | KIAA1833-Homo sapiens (Human)- |
| NO: 48 | | | [K1833_HUMAN] |
| SEQ | Q9NVA4 | LVSIVVAVPL | Transmembrane protein 34-Homo |
| ID | | | sapiens (Human)-[TMM34_HUMAN] |
| NO: 49 | | | |
| SEQ | O43909 | LWPDIGVPI | Exostosin-like 3-Homo sapiens |
| ID | | | (Human)-[EXTL3_HUMAN] |
| NO: 50 | | | |
| SEQ | Q8WZ69 | MDRVLLHV | CK040_HUMAN Putative uncharacterized |
| ID | | | protein C11orf40 (Ro/SSA1-related |
| NO: 51 | | | protein) |
| SEQ | Q92543 | MEAAMKGLVQE | Sorting nexin-19-Homo sapiens |
| ID | | | (Human)-[SNX19_HUMAN] |
| NO: 52 | | | |
| SEQ | P12956 | MGFKPLVL | ATP-dependent DNA helicase 2 subunit |
| ID | | | 1-Homo sapiens (Human)- |
| NO: 53 | | | [KU70_HUMAN] |
| SEQ | Q9H799 | MLDLHCDKI | Uncharacterized protein C5orf42- |
| ID | | | Homo sapiens (Human)-[CE042 HUMAN] |
| NO: 54 | | | |
| SEQ | Q9Y6L6 | MTYDGNNPVT | Solute carrier organic anion |
| ID | | | transporter family member 1B1-Homo |
| NO: 55 | | | sapiens (Human)-[SO1B1 HUMAN] |
| SEQ | Q9H5V7 | MVDNPLNQLS | Zinc finger protein Pegasus-Homo |
| ID | | | sapiens (Human)-[IKZF5_HUMAN] |
| NO: 56 | | | |
| SEQ | O75319 | NKDNDKLI | DUS11_HUMAN RecName: Full = RNA/RNP |
| ID | | | complex-1-interacting phosphatase; |
| NO: 57 | | | AltName: Full = Phosphatase that |
| | | | interacts with RNA/RNP complex 1; |
| | | | AltName: Full = Dual specificity |
| | | | protein phosphatase 11 |
| SEQ | Q99650 | NKEVEEERIAG | Oncostatin-M specific receptor |
| ID | | | subunit beta precursor-Homo sapiens |
| NO: 58 | | | (Human)-[OSMR_HUMAN] |
| SEQ | Q08AD1 | QALAQKGLYVT | Calmodulin-regulated spectrin- |
| ID | | | associated protein 1-like protein 1- |
| NO: 59 | | | Homo sapiens (Human)-[CA1L1_HUMAN] |
| SEQ | P50789 | QINAMNSDILE | Major capsid protein L1-Human |
| ID | | | papillomavirus type 23-[VL1_HPV23] |
| NO: 60 | | | |
| SEQ | O75381 | QINEQVEKL | Peroxisomal membrane protein PEX14- |
| ID | | | Homo sapiens (Human)-[PEX14_HUMAN] |
| NO: 61 | | | |
| SEQ | Q8IVF4 | QLDELNQKL | Dynein heavy chain 10, axonemal- |
| ID | | | Homo sapiens (Human)-[DYH10_HUMAN] |
| NO: 62 | | | |
| SEQ | Q9BUI4 | QVHKRGVVEYEA | RPC3_HUMAN RecName: Full = DNA-directed |
| ID | | | RNA polymerase III subunit RPC3; |
| NO: 63 | | | Short = RNA polymerase III subunit C3; |
| | | | AltName: Full = DNA-directed RNA |
| | | | polymerase III subunit C; AltName: |
| | | | Full = DNA-directed III 62 kDa |
| | | | polypeptide; AltName: Full = RPC62 |
| SEQ | Q8TC76 | RIIKWLYSI | Protein FAM110B-Homo sapiens |
| ID | | | (Human)-[F110B_HUMAN] |
| NO: 64 | | | |
| SEQ | Q91YB0 | RINTVSLKEA | Circadian locomoter output cycles |
| ID | | | protein kaput-Homo sapiens (Human)- |
| NO: 65 | | | [CLOCK_HUMAN] |
| SEQ | O60508 | RLFPLSGHLLL | Pre-mRNA-processing factor 17-Homo |
| ID | | | sapiens (Human)-[PRP17_HUMAN] |
| NO: 66 | | | |
| SEQ | Q96AB3 | RLLEVPVML | ISOC2_HUMAN Isochorismatase domain- |
| ID | | | containing protein 2, mitochondrial |
| NO: 67 | | | precursor |
| SEQ | Q15022 | SIMSIDKAVT | Polycomb protein SUZ12-Homo sapiens |
| ID | | | (Human)-[SUZ12_HUMAN] |
| NO: 68 | | | |
| SEQ | P60019 | SLDRYIKI | GPR34_PANTR RecName: Full = Probable G- |
| ID | | | protein coupled receptor |
| NO: 69 | | | 34gi\|52000690\|sp\|Q6XCF2.1\|GPR34_GORGO |
| | | | RecName: Full = Probable G-protein |
| | | | coupled receptor |
| | | | 34gi\|82592894\|sp\|Q3SAG9.1\|GPR34_PANPA |
| | | | RecName: Full = Probable G-protein |
| | | | coupled receptor |
| | | | 34gi\|12643337\|sp\|Q9UPC5\|GPR34_HUMAN |
| | | | Probable G-protein coupled receptor |
| | | | 34 |
| SEQ | Q9BVK6 | SLFAGGMLRV | Transmembrane emp24 domain-containing |
| ID | | | protein 9 precursor-Homo sapiens |
| NO: 70 | | | (Human)-[TMED9_HUMAN] |
| SEQ | Q9ULM0 | SLMQCWQL | Pleckstrin homology domain-containing |
| ID | | | family H member 1-Homo sapiens |
| NO: 71 | | | (Human)-[PKHH1_HUMAN] |
| SEQ | Q8N3G9 | SLVAKDNGSL | Transmembrane protein 130 precursor- |
| ID | | | Homo sapiens (Human)-[TM130_HUMAN] |
| NO: 72 | | | |
| SEQ | Q8IZF5 | SLVLRKLDHL | Probable G-protein coupled receptor |
| ID | | | 113 precursor-Homo sapiens (Human)- |
| NO: 73 | | | [GP113 HUMAN] |
| SEQ | Q5VT52 | SPALALPNLAN | Uncharacterized protein KIAA0460- |
| ID | | | Homo sapiens (Human)-[K0460_HUMAN] |
| NO: 74 | | | |
| SEQ | Q92959 | SSSGLISSLNEI | SO2A1_HUMAN Solute carrier organic |
| ID | | | anion transporter family member 2A1 |
| NO: 75 | | | (Solute carrier family 21 member 2) |
| | | | (Prostaglandin transporter) (PGT) |
| SEQ | Q5GLZ8 | TCFNLLDL | HERC4_HUMAN Probable E3 ubiquitin- |
| ID | | | protein ligase HERC4 (HECT domain and |
| NO: 76 | | | RCC1-like domain-containing protein |
| | | | 4) |
| SEQ | Q9NT68 | TLTVGTNGGLK | Teneurin-2-Homo sapiens (Human)- |
| ID | | | [TEN2_HUMAN] |
| NO: 77 | | | |
| SEQ | P16220 | TLVQLPNGQTV | cAMP response element-binding protein- |
| ID | | | Homo sapiens (Human)- |
| NO: 78 | | | [CREB1_HUMAN] |
| SEQ | Q5GLZ8 | TVFVLDDGTV | Probable E3 ubiquitin-protein ligase |
| ID | | | HERC4-Homo sapiens (Human)- |
| NO: 79 | | | [HERC4_HUMAN] |
| SEQ | Q8NF91 | TVMMGKKL | Nesprin-1-Homo sapiens(Human)- |
| ID | | | [SYNE1_HUMAN] |
| NO: 80 | | | |
| SEQ | Q8WTV0 | VLGAVMIVMV | Scavenger receptor class B member 1- |
| ID | | | Homo sapiens (Human)-[SCRB1_HUMAN] |
| NO: 81 | | | |
| SEQ | Q96PD2 | VLVPVLVMV | Discoidin, CUB and LCCL domain- |
| ID | | | containing protein 2 precursor-Homo |
| NO: 82 | | | sapiens (Human)-[DCBD2_HUMAN |
| SEQ | P23470 | VMLPDNQSL | Receptor-type tyrosine-protein |
| ID | | | phosphatase gamma precursor-Homo |
| NO: 83 | | | sapiens (Human)-[PTPRG_HUMAN] |
| SEQ | Q9BYE9 | YINQSKAIDYEA | PCD24_HUMAN RecName: |
| ID | | | Full = Protocadherin-24; AltName: |
| NO: 84 | | | Full = Protocadherin LKC; Short = PC-LKC; |
| | | | Flags: Precursor |
| SEQ | O60885 | YLLRVVLKT | Bromodomain-containing protein 4- |
| ID | | | Homo sapiens (Human)-[BRD4_HUMAN] |
| NO: 85 | | | |
| SEQ | Q13813 | YVEFTRSLFVN | Spectrin alpha chain, brain-Homo |
| ID | | | sapiens (Human)-[SPTA2_HUMAN] |
| NO: 86 | | | |

In certain cases, the epitopic peptide(s) may comprise or consist of an amino acid sequence selected from the group consisting of:

(SEQ ID NO: 82)
VLVPVLVMV;
(SEQ ID NO: 29)
KIYQWINEL;
(SEQ ID NO: 33)
KLGEFAKVLEL;
(SEQ ID NO: 19)
GMYGKIAVMEL;
(SEQ ID NO: 34)
KLIPFLEKL;
and
(SEQ ID NO: 67)
RLLEVPVML.

The following is presented by way of example and is not to be construed as a limitation to the scope of the claims.

### EXAMPLES

### Example 1

### Synthesis and Characterisation of Nanoparticles

Test ligands and their identification numbers are given below (Molecular wt);

(SEQ ID NO: 87)
SIINFEKL (963)
SIINFEKL-N-(CH₂)₂-SH (1021)
(SEQ ID NO: 88)
FLSIINFEKL-N-(CH₂)₂-SH (1280)
(SEQ ID NO: 89)
FLAAYSIINFEKL-N-(CH₂)₂-SH (1587)
(SEQ ID NO: 90)
AAYSIINFEKL-N-(CH₂)₂-SH (1325)
HS(CH₂)₂-CONH-SIINFEKL (1051)
HS(CH₂)₂-CONH-FLSIINFEKL (1309)
HS(CH₂)₂-CONH-FLAAYSIINFEKL (1616)
HS(CH₂)₂-CONH-AAYSIINFEKL (1356)
HS-(CH₂)₁₀-(CH₂OCH₂)₇-CONH-SIINFEKL (1471)
HS-(CH₂)₁₀-(CH₂OCH₂)₇-CONH-FLSIINFEKL (1732)
HS-(CH₂)₁₀-(CH₂OCH₂)₇-CONH-FLAAYSIINFEKL (2034)
HS-(CH₂)₁₀-(CH₂OCH₂)₇-CONH-AAYSIINFEKL (1774)

Test NPs were synthesized using 10 μmole Gold Chloride (Aldrich 484385), 30 μmole glucose with a thio ethyl linker (GlcC2) and 1.5 μmole of peptide ligand (variable 1.5-3 mg).

The following method was used; 1.5 μmole peptide was dissolved in 2 ml methanol, followed by the addition of 30 μmole GlcC2 in 200 μl methanol, and 116 μl of aqueous gold chloride containing 10 μmole Au. The sample was vortexed for 30 sec, shaken for approximately 5 min and then under rapid vortexing for a total of 30 sec 200 μl of 1M NaBH₄ was added, tubes were sealed and then gently shaken for 1.5 h.

Samples were bench spun the supernatant removed and the dark pellet dissolved in 2 ml water, and then transferred to a 10 kDa vivaspin for a total of 4 times 2 ml water washes each of 8 min at 5 Krpm. Nanoparticles (NPs) were removed from the vivaspins and made up to 500 μl with water, they were then subjected to 15 Krpm bench spin to remove any large aggregates.

Gold content post spin/production by in house assay is shown below 100% yield would be 1.97 mg;

| NP | Total mg Au |
|---|---|
| 2 | 1.14 |
| 3 | 0.03 |
| 4 | 0.00 |
| 5 | 0.05 |
| 6 | 1.34 |
| 7 | 1.06 |
| 9 | 1.59 |
| 10 | 1.69 |
| 11 | 1.37 |
| 13 | 1.49 |
| Glc | 1.02 |

NPs 3, 4 and 5 showed large near complete aggregation, addition of DMSO to these aggregates failed to solubilise these particle.

The remaining NP samples were respun to remove any further aggregates, and aliquots taken to allow for later analyses, specifically gold and peptide content. One aliquot was made up to 500 μl with water, dated and labelled, these were subsequently used for HLA presentation testing.

The final analysis of these 500 μl samples for HLA presentation was as follows;

| | Total μmole | | nmole |
|---|---|---|---|
| NP | Au | nmole peptide* | peptide** |
| 2 | 1.46 | 166 | 106 |
| 3 | — | — | — |
| 4 | — | — | — |
| 5 | — | — | — |
| 6 | 3.09 | 348 | 263 |
| 7 | 3.36 | 287 | 219 |
| 9 | 4.77 | 508 | 286 |
| 10 | 4.42 | 256 | 372 |
| 11 | 3.26 | 235 | 830 |
| 13 | 4.19 | 386 | 1083 |
| Glc | 3.11 | nd | nd |

*BSA Std
**Peptide Std

Ligand ratios used were such that 2 peptides should theoretically be attached to each NP of approximately 100Au atoms, the data above suggests approximately 6-8 peptides/100Au atoms. This could simple be an artefact of BCA method (and BSA standard) used for peptide measurement of NP bound peptides or perhaps the peptide ligands attach.

Analysis of peptide content is both crucial and in this case complex, the BCA method was used. Unfortunately gold NPs exhibit large uv/vis absorbance, so in addition to running the test samples aliquots of NPs were also measured in water and blanked against water to determine their absorbance at 565 nm the wavelength used in the BCA assay, this absorbance amounted to approximately 20% of the test sample BCA assay value and was individually corrected for. This correction however, assumes that a peptide NP subjected to BCA analysis will still maintain the same absorbance seen for the free NP on top of the BCA specific component; this is consistent with the high extinction coefficients seen with just pure gold NPs.

In the table above * refers to quantitation related to a BSA standard initially on a weight basis then converted to moles of peptide, in the ** column the individual peptide ligands were used as standards.

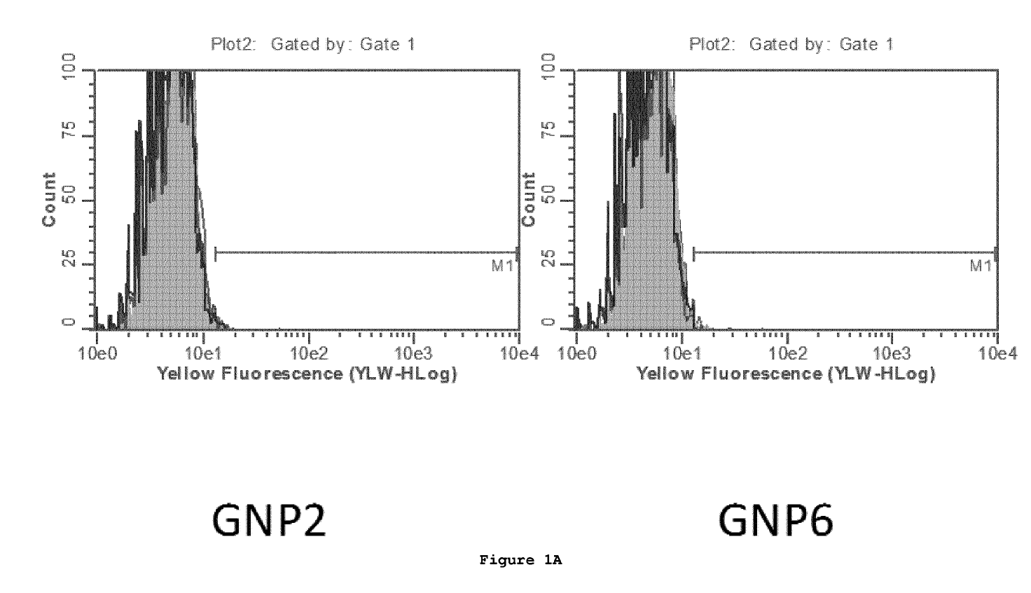

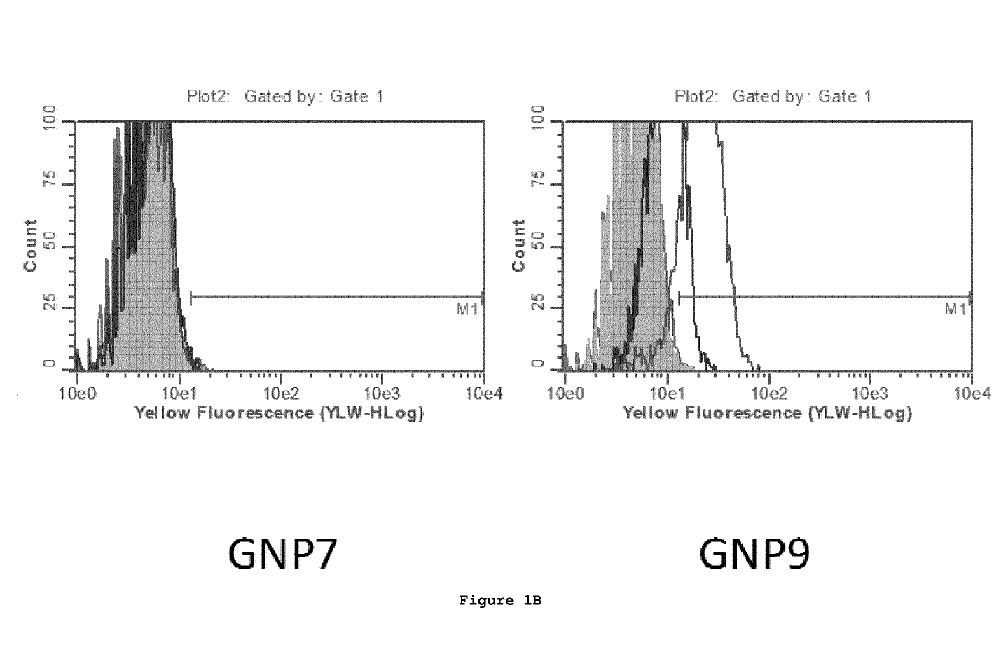

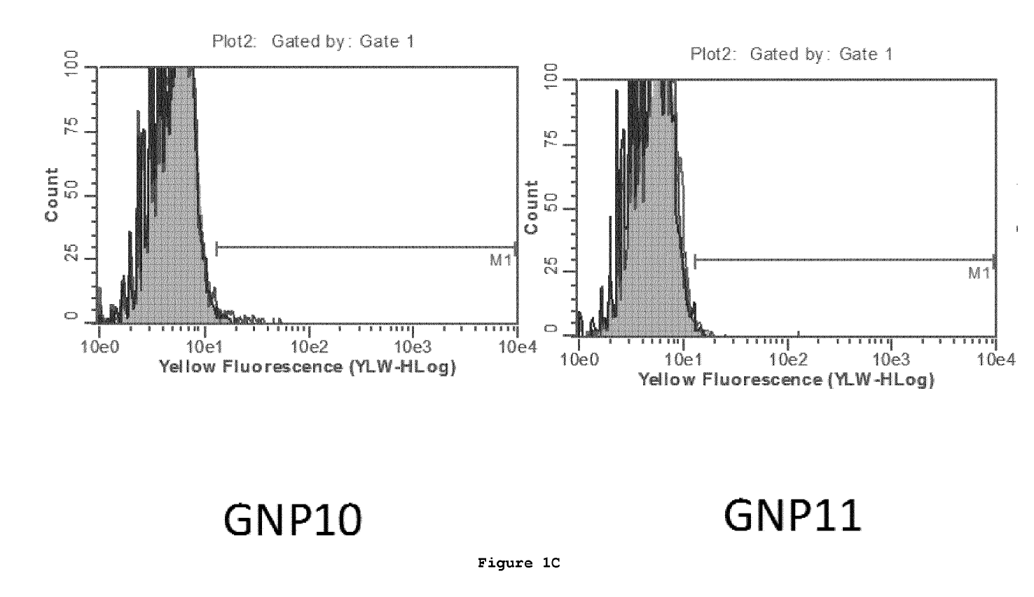

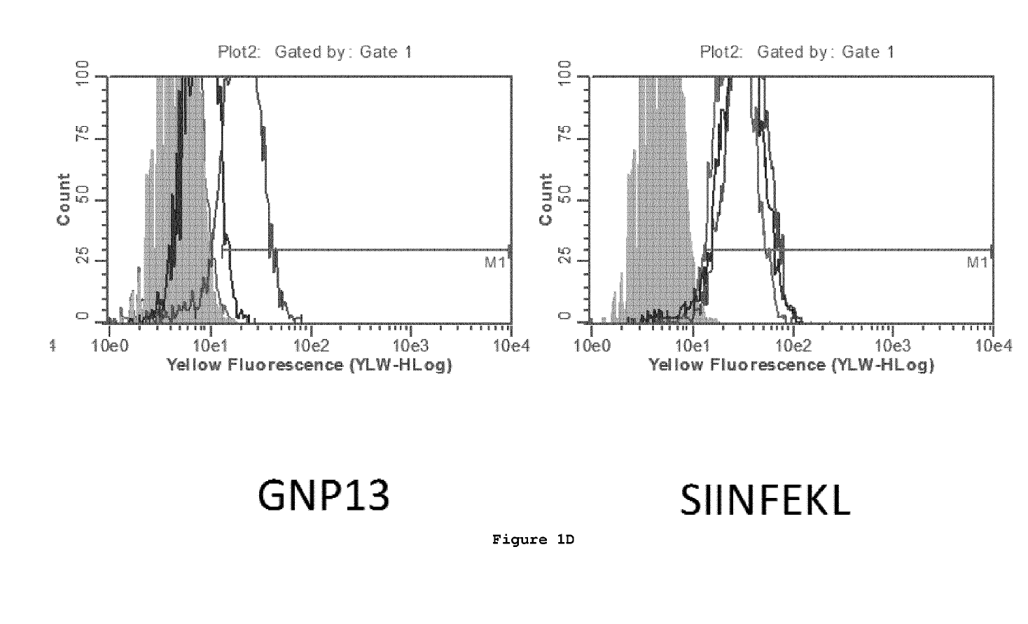

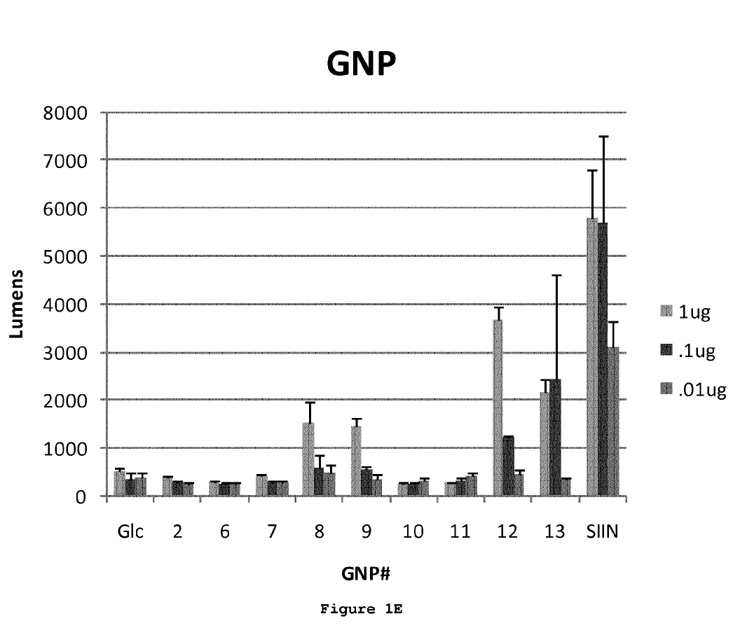

Initial analyses using Sephadex G-50 with PBS elution to try to resolve free form NP bound peptide was performed primarily with NP6, suggest that little/no free ligand is contaminating the NP preparations. Iodine was used to release all NP bound ligands the iodine began to appear in the fractions 16+ for FIG. 1.

Larger fraction sizes and equivalent amounts of NP pre and post iodine treatment were then used, the iodine released peptide elutes with a peak in fraction 10, this material appears smaller than the standard peptide possibly because the latter is oxidized/dimeric. Individual corrections were also applied for non peptidic absorbances from NP6, near equivalent areas under the curve were obtained for NP6 corrected and NP6+ iodine.

### Production of NP8 and 12

NP8 and 12 were successfully produced by the methods above, quantitations given below represent the 500 μl sample subsequently used, which in turn is (60% of the total preparation);

| | | nmole |
|---|---|---|
| NP | μmole Au | peptide* |
| 8 | 5.28 | 175 |
| 12 | 3.21 | 65 |

*Determined by Coomassie method

### Repeat Production of NPs 2-5

NP's 2-5 were produced by a variation using 75% methanol not 95% in the synthesis stage. NP2 produced a ‘normal’ NP as previously, NPs 3, 4 and 5 as before formed aggregates, these aggregates where not soluble in water, 10% acetic acid, PBS, DMSO or DMF, however 300 mM NaOH did result in total NP solubilisation.

Additional synthesis was carried out using Sephadex G-50 to remove any free peptide. These NPs were made as above but with the following changes:
The free peptides were not fully soluble in MeOH so in addition to the 2 ml MeOH 100 μl water was added plus for P8 70 μl of 1MNaOH and for P9 only 20 μl NaOH that was required to effect full peptide solubilisation. NaOH is compatible with NP synthesis, the water and alkali reduced the final MeOH % post borohydride reduction to 82 and 83.5% for NP8 and NP9 respectively.
An extra wash step was included after the initial spin down post reduction but pre-vivaspin, this was performed with 2 ml MeOH and 100 μl of 1MNaCl.
Post vivaspin half of each preparation was subjected to a Sephadex G-50 column eluted with PBS, fractions collected and aliquots measured for protein by BCA, tight pools of the main proteinaceous peaks were pooled and then resubjected to vivaspin with water washes to effect solvent change.

Only half of the NP preparation was passed down G-50, the resultant profiles and pools were found to be essentially clear of free peptide. The NP brown colouration could be visually seen up to fraction 12, in a separate run 50 ul of the stock preparation was rerun under the same conditions and the 515 nm absorbance measured (which will detect Au NPs not free peptides) and gives an indication if the NP is trailing off the G-50 column.

The final pooled material had the following specifications;

| | NP8 | NP9 |
|---|---|---|
| Volume ml | 0.5 | 0.5 |
| Peptide by BCA (BSA | 638 | 490 |
| std) μg/ml | | |
| Theoretical peptide | 225 | 118 |
| μg/ml | | |
| Au mg/ml | 1.30 | 0.81 |

The theoretical values are determined by assuming 44 ligands/100Au, random competition between the two ligands at the time of NP formation and the Au yield.

NP Ligand Release with Iodide

An aliquot of NP9 was mixed with a 4-fold volume excess of 1M KI and left at 4° C. for 4 days, in order to result in complete ligand release. After 4 days, the material was centrifuged and the clear supernatant passed down G-50 and fractions collected and assayed by BCA. The amount of NP9 post KI assayed was exactly twice that used for the NP9 alone (a correction of 1.1 was applied for inter-assay absorbance differences), the areas were found to be almost exactly 2:1 suggesting complete ligand removal. The amount of ligand released was quantitated using 2 assays; Coomassie and BCA in conjunction with 2 standards peptide 9 and BSA, and is tabulated below.

| Protein std used | Coomassie μg | BCA μg |
|---|---|---|
| BSA | 377 | 611 |
| P9 | 2210 | 1062 |

The data in the table has been corrected up to the total expected yield for the whole preparation.

In conclusion, peptide-containing NPs have been synthesized. The peptide NPs are essentially devoid of contaminating free peptides by simple use of Sephadex G-50 gel filtration chromatography. Iodide was successfully used to release NP bound peptide, and gave quantitative yield data.

### Example 2

### Evaluation of Presentation Assays

T cell receptors (TCR) are on the surface of T lymphocytes and recognize peptides in the context of major histocompatibility complex (MHC) (1). Generally, antigen presenting cells (APC) contain machinery to process proteins and load them onto empty MHC. While CD4+ T cells recognize MHC Class II (MHCII), CD8+ T cells respond to MHC Class I (MHCI). Conventionally, MHCII peptides derive from endocytosed components of the extracellular milieu. In contrast, MHCI loads peptides processed from an intracellular source (1, 2).

SIINFEKL (SEQ ID NO: 87), a peptide epitope that is derived from ovalbumin (OVA), is presented in the context of a murine MHCI allele termed H-2K^{b} (3). If OVA is expressed in a murine cell expressing H-2K^{b}, SIINFEKL (SEQ ID NO: 87) is presented conventionally. However, if OVA is supplied exogenously, SIINFEKL can be presented by an alternative process known as MHCI cross-presentation (4, 5). In fact, haplotype-matched mouse immunized with OVA generate an immunodominant response to SIINFEKL (SEQ ID NO: 87).

Therefore, many reagents have been developed to assay the presentation of this peptide in an effort to further the understanding of conventional and alternative MHCI presentation. These reagents can be utilized to experiment with the potential chemical linkages of peptides in nanoparticles. Thus, we can uncover which linkage is most easily processed in a mouse cell line.

### Results and Discussion

### Flow Cytometry as a Measure of Presentation

In order to analyze epitope linkage to nanoparticles, we first optimized a readout assay for the presentation of the epitope released from the nanoparticle. For this purpose, SIINFEKL (SEQ ID NO: 87) presentation in LK^{b} cells was evaluated. As mentioned earlier, more than one reagent exists. Of the two methods tested, one is flow cytometry-based, while the other is cell-based. The flow cytometry-based method begins with pulsing LK^{b} cells with differing amounts of SIINFEKL (SEQ ID NO: 87) peptide. After allowing enough time for peptide binding to surface K^{b} molecules, cells were washed and incubated with the 25.D1.16 antibody which is specific to the SIINFEKL:K^{b} complex. Next, the cells were secondarily labelled and subjected to flow cytometry analysis using unpulsed cells as the background reading. It was found that this method detected surface complexes when the cells were pulsed with as little as 5 ng/mL.

### T Cell Activation as a Measure of Antigen Presentation

Another form of epitope-specific antigen presentation is the measurement of T cell activation by the MHCI peptide complex. Here, we used B3Z (OVA peptide specific T cell line), which recognizes SIINFEKL (SEQ ID NO: 87) the context of K^{b}. As a convenient measure, this T cell line contains β-galactosidase cloned with the NFAT promoter. Upon peptide recognition and T cell activation, β-galactosidase is expressed and conversion of a detectible substrate serves as an excellent measure of antigen presentation to T cells.

To evaluate this method, we performed a similar experiment as above. LK^{b} cells were pulsed with SIINFEKL peptide, washed, and then co-incubated with B3Z T cell line overnight. The next day, cells were lysed and β-galactosidase was measured using a luminescent substrate. As expected, the resolution of this method was similar to the previous method with the limit of detection at approximately 5 ng/mL.

Therefore, both methods exhibit approximately the same resolution and were selected for use in the evaluation of nanoparticle-peptide constructs.

### Materials and Methods

### Cell Lines

LK^{b} cells are mouse fibroblasts and were the primary line used. Specifically, they are L929 cells stably expressing the murine H-2K^{b} molecule.

### Synthetic Peptides

Synthetic SIINFEKL (OVA 257-264) (SEQ ID NO: 87) peptides were purchased from Genscript USA (Piscataway, N.J.). Peptides were resuspended to 5 mg/mL in DMSO and pulsed onto cells at the concentration denoted in the figures.

### T Cell Hybridomas

The SIINFEKL:Kb-specific T hybridoma (B3Z) expresses β-galactosidase upon recognition of peptide-MHC class I complexes and has been described previously (3, 6). T cell hybridomas were maintained in complete RPMI plus 10% FCS and 0.05 mM 2-ME. Activation was measured using the luminescent substrate Galactolight Plus (Applied Biosystems, Foster City, Calif.) according to the manufacturer's instructions. Light intensity was measured using a TopCount NXT plate reader (Perkin Elmer, Waltham, Mass.).

### Flow Cytometry

LK^{b} cells were treated with varying amounts of SIINFEKL (SEQ ID NO: 87) peptides. After a 2 hr incubation, cells were collected, washed once with PBS, and then incubated for 1 hr with 25.D1.16 culture supernatant (monoclonal antibody specific for SIINFEKL complexed to H-2K^{b}) (7) on ice. Cells were then washed two times in PBS and incubated for 30 min on ice with FITC-labeled goat anti-mouse IgG secondary antibody (Caltag Laboratories, Burlingham, Calif.). Finally, cells were washed two times with PBS and resuspended in PBS+0.1% BSA for flow cytometry on a Guava EasyCyte Plus Flow Cytometer (Millipore, Billerica, Mass.) and analyzed using the accompanying software.

### Antigen Presentation Assays

To assay SIINFEKL (SEQ ID NO: 87) presentation using a flow cytometric or cell-based method, we pulsed LK^{b} cells in a 15 mL conical at 37° C. for 2 hrs. Following this incubation, cells were washed once with PBS and then subjected to detection using flow cytometry or added to B3Z cells at an effector to target ratio of 1:1.

### REFERENCES

1. Vyas, J. M., A. G. Van der Veen, and H. L. Ploegh. 2008. The known unknowns of antigen processing and presentation. Nature reviews 8:607-618.
2. Hansen, T. H., and M. Bouvier. 2009. MHC class I antigen presentation: learning from viral evasion strategies. Nature reviews 9:503-513.
3. Shastri, N., and F. Gonzalez. 1993. Endogenous generation and presentation of the ovalbumin peptide/K^{b} complex to T cells. Journal of Immunology 150:2724-2736.
4. Amigorena, S., and A. Savina. 2010. Intracellular mechanisms of antigen cross presentation in dendritic cells. Current opinion in immunology 22:109-117.
5. Blanchard, N., and N. Shastri. 2010. Cross-presentation of peptides from intracellular pathogens by MHC class I molecules. Annals of the New York Academy of Sciences 1183:237-250.
6. Tewari, M. K., G. Sinnathamby, D. Rajagopal, and L. C. Eisenlohr. 2005. A cytosolic pathway for MHC class II-restricted antigen processing that is proteasome and TAP dependent. Nature immunology 6:287-294.
7. Porgador, A., J. W. Yewdell, Y. Deng, J. R. Bennink, and R. N. Germain. 1997. Localization, quantitation, and in situ detection of specific peptide-MHC class I complexes using a monoclonal antibody. Immunity 6:715-726.

### Example 3

### Nanoparticle-Peptide Presentation Assays

The test ligands listed below were constructed and attached to gold nanoparticles (GNP) by the above-described linker chemistry.

(SEQ ID NO: 87)
1. SIINFEKL
2. SIINFEKL-N-(CH2)2-SH
(SEQ ID NO: 88)
3. FLSIINFEKL-N-(CH2)2-SH
(SEQ ID NO: 89)
4. FLAAYSIINFEKL-N-(CH2)2-SH
(SEQ ID NO: 90)
5. AAYSIINFEKL-N-(CH2)2-SH
6. HS(CH2)2-CONH-SIINFEKL
7. HS(CH2)2-CONH-FLSIINFEKL
8. HS(CH2)2-CONH-FLAAYSIINFEKL
9. HS(CH2)2-CONH-AAYSIINFEKL
10. HS-(CH2)10-(CH2OCH2)7-CONH-SIINFEKL
11. HS-(CH2)10-(CH2OCH2)7-CONH-FLSIINFEKL
12. HS-(CH2)10-(CH2OCH2)7-CONH-FLAAYSIINFEKL
13. HS-(CH2)10-(CH2OCH2)7-CONH-AAYSIINFEKL

SIINFEKL (SEQ ID NO: 87), an epitope derived from ovalbumin that is presented in the context of the murine MHCI molecule H-2 Kb, was measured using two methods. One method utilized a TCR-like antibody termed 25.D1.16, also referred to as “Angel”, that recognize SIINFEKL/MHCI complex. In addition, we assessed presentation using the B3Z, SIINFEKL (SEQ ID NO: 87) peptide specific CTL hybridoma, which expresses beta-galactosidase under the NFAT (CTL signaling molecule) promoter, which upon activation express beta-gal measured by a light emitting substrate.

### A. Analysis of GNP

To assess the ability of cells to process and present SIINFEKL (SEQ ID NO: 87) associated with GNP, we used L-Kb murine fibroblasts. As demonstrated in FIG. 1, GNPs 8, 9, 12, and 13 displayed good processing and presentation. This was found to be the case for both flow cytometric (FIG. 1A-D—processing) and CTL-based methods (FIG. 1E—presentation). Thus, FLAAYSIINFEKL (SEQ ID NO: 89) and AAYSIINFEKL (SEQ ID NO: 90) exhibited superior in vitro SIINFEKL (SEQ ID NO: 87) processing (the underlined portion showing the peptide portion of the linker. Also, as illustrated in FIG. 1E, HS—(CH2)10-(CH2OCH2)7-CONH chemistry was found to be better processed than HS(CH2)2-CONH. However, HS(CH2)2-CONH was still found to be processed very efficiently. Additionally, we note a dose-dependent reduction of presentation with no detection at 0.01 μg/mL.

### B. Analysis of Free Peptide

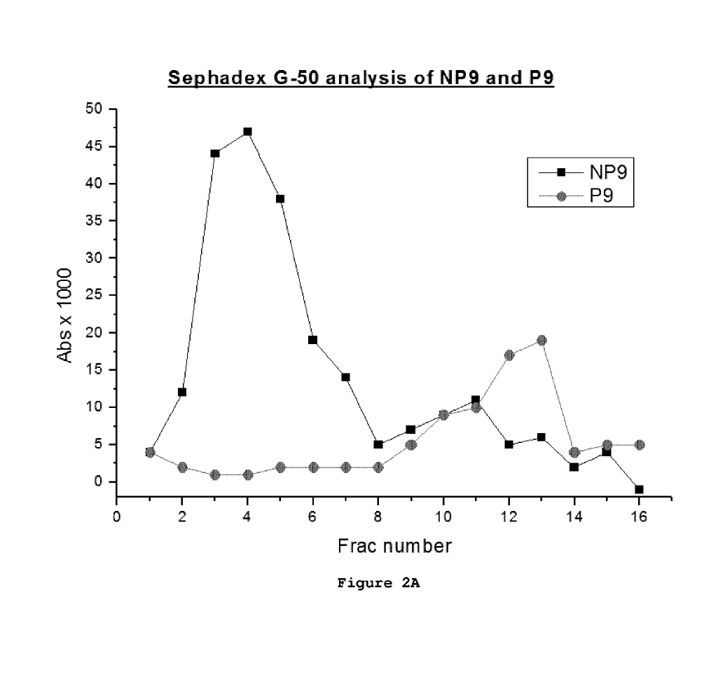

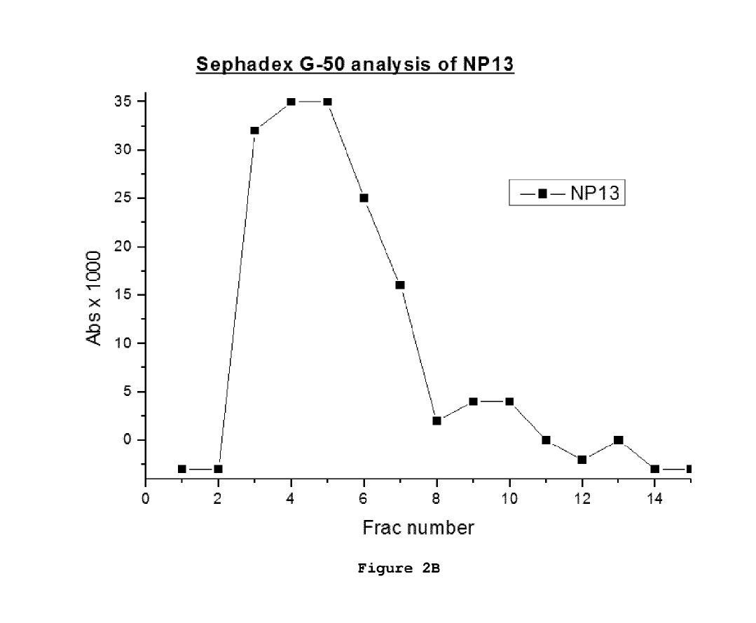

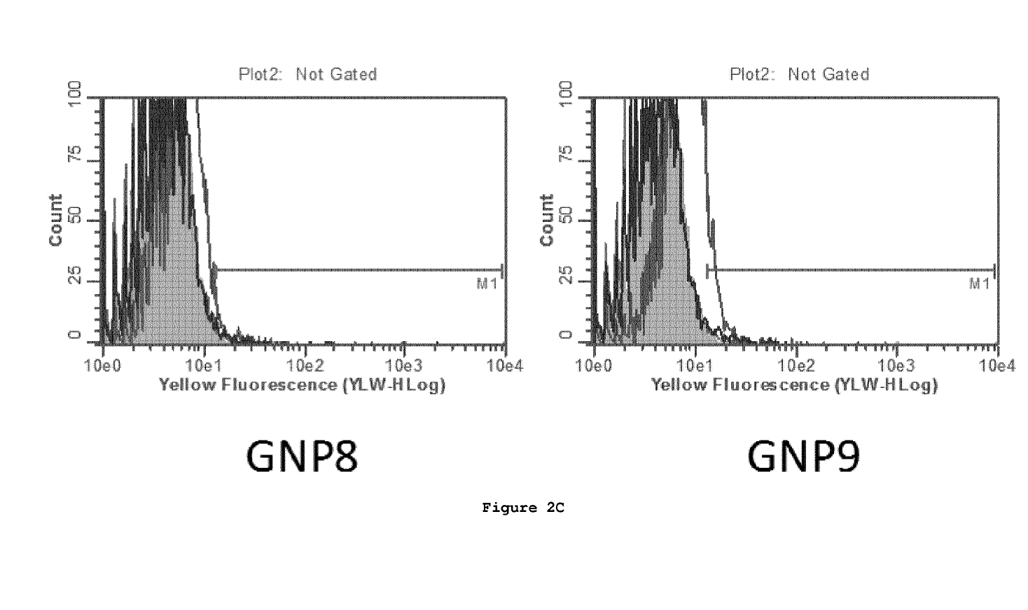

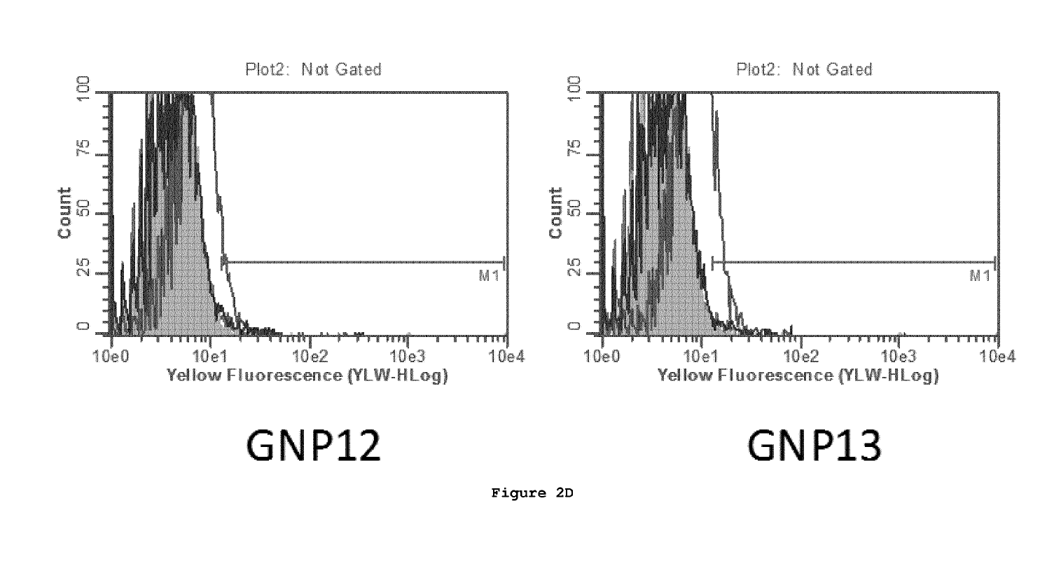

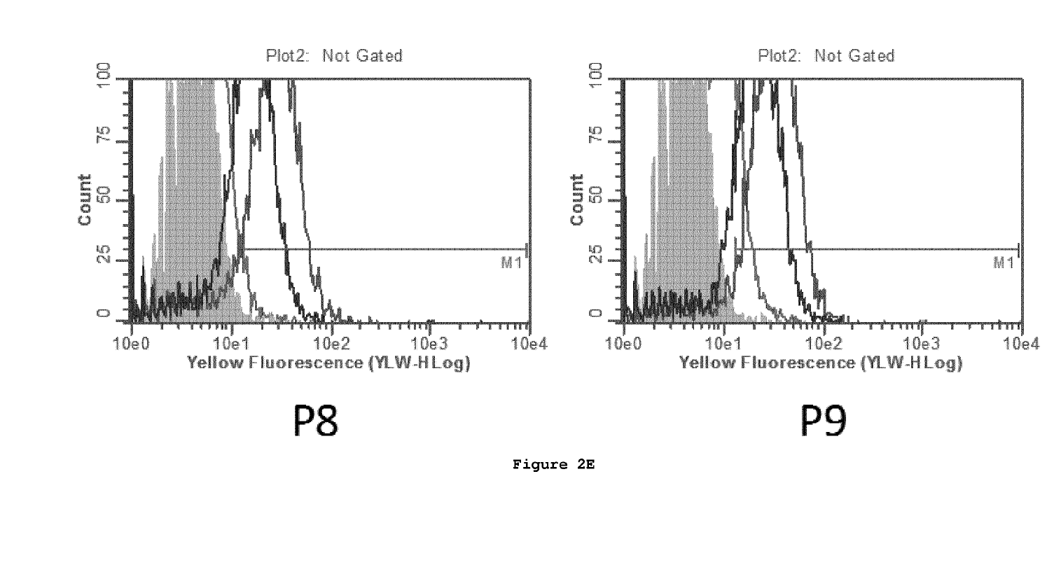

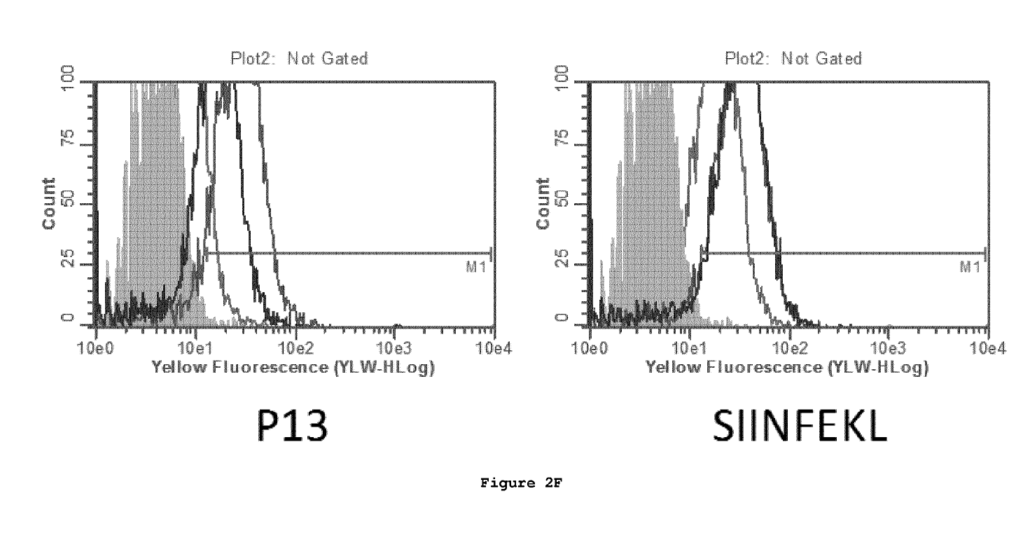

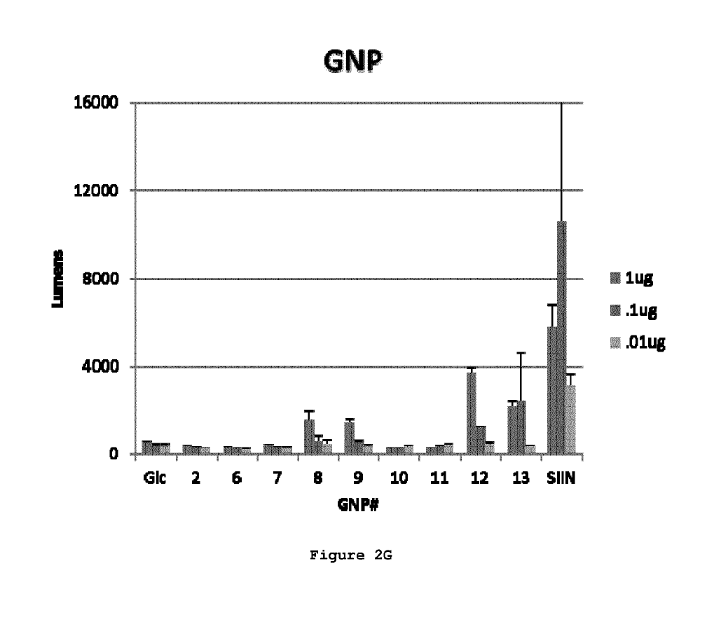

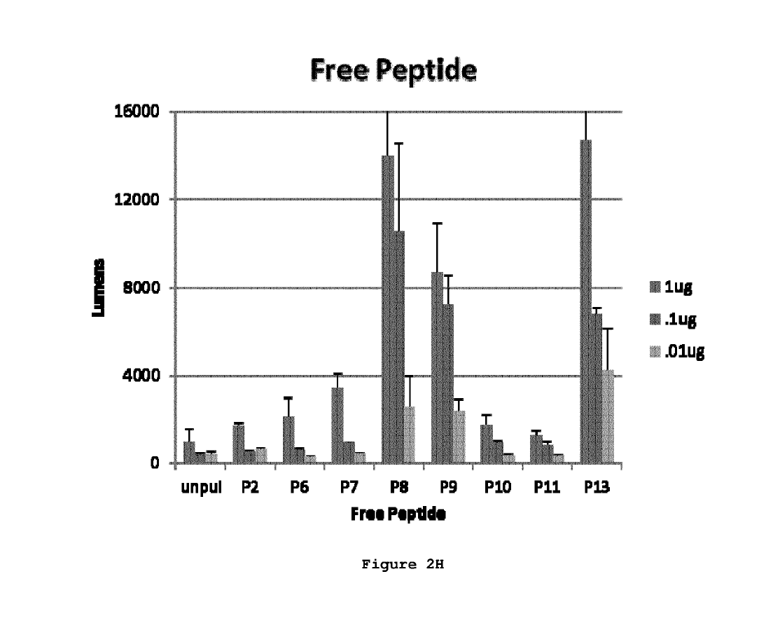

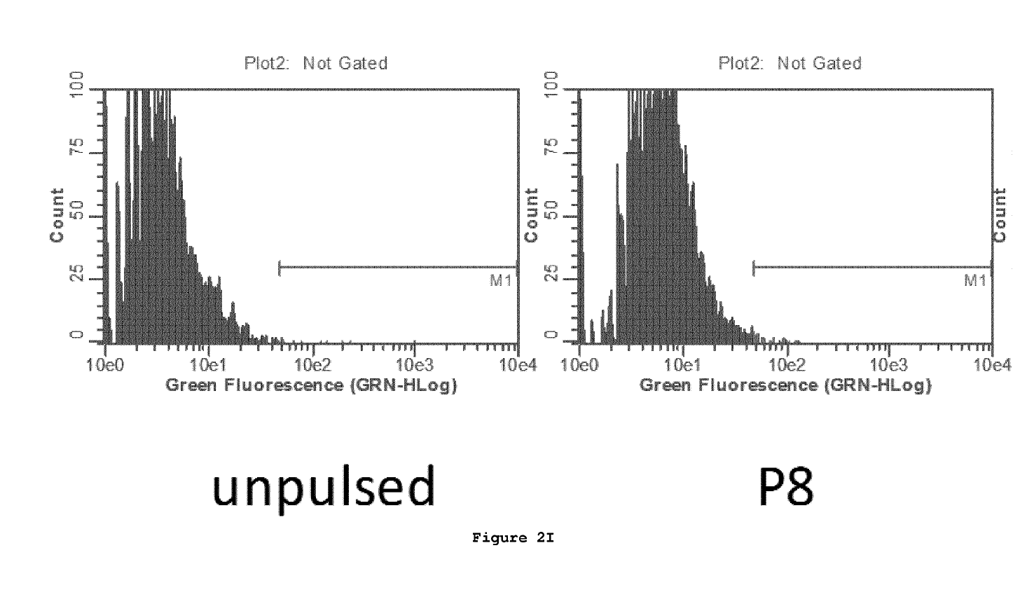

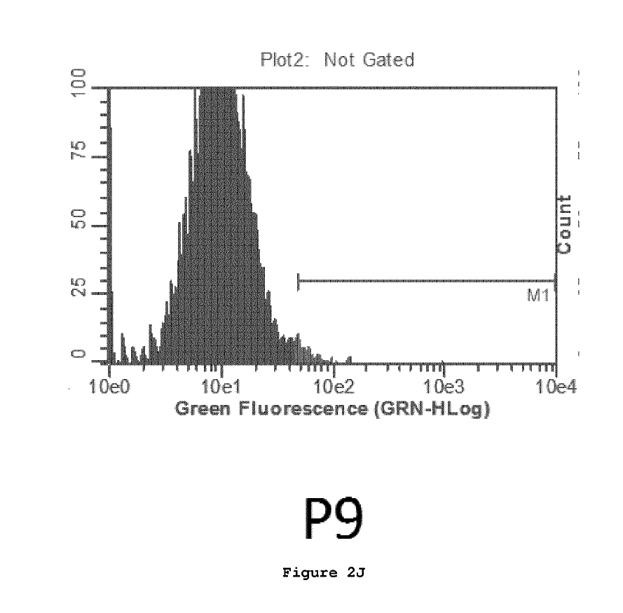

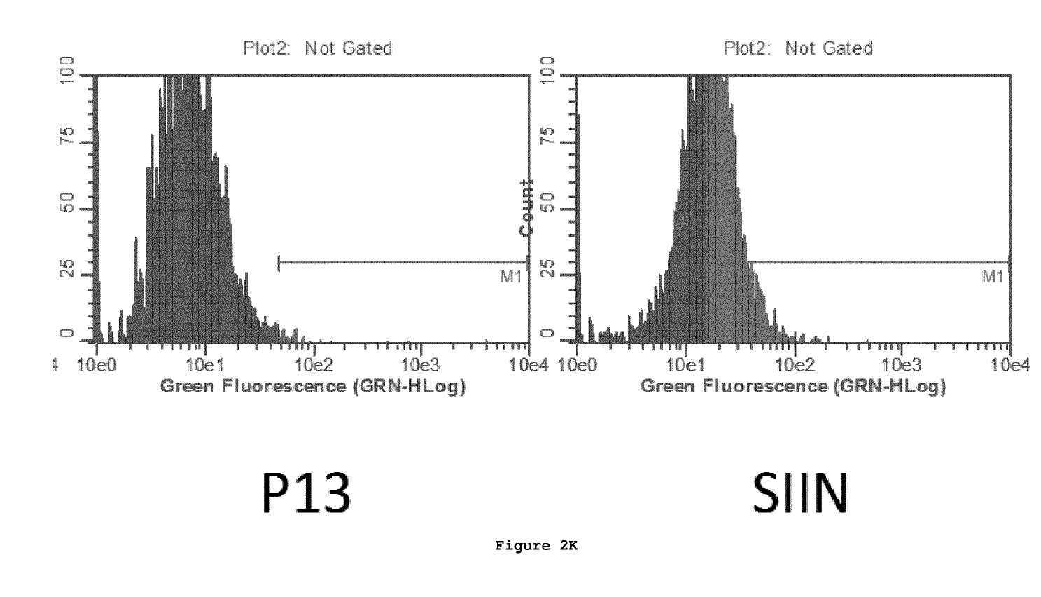

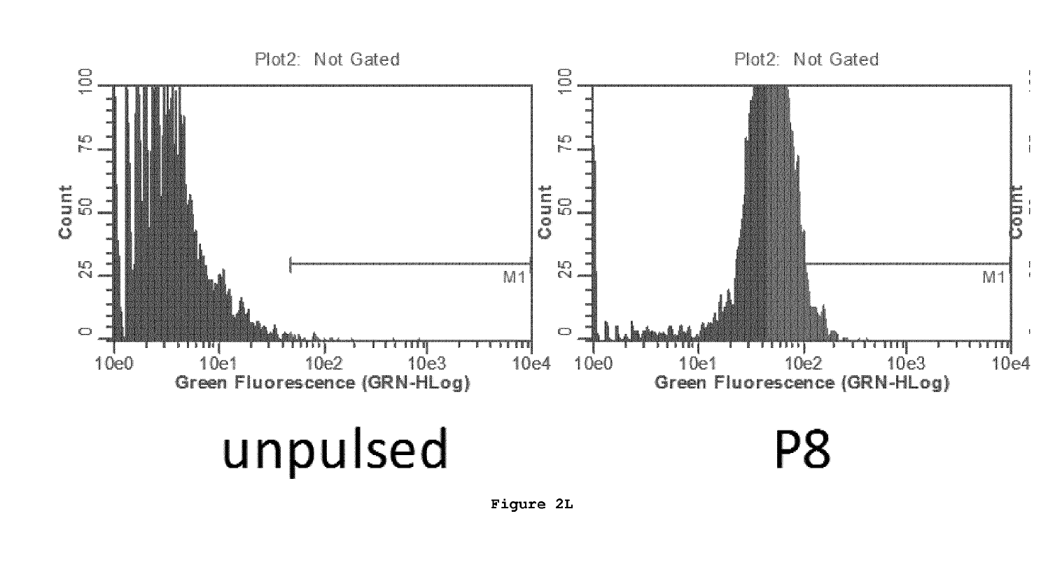

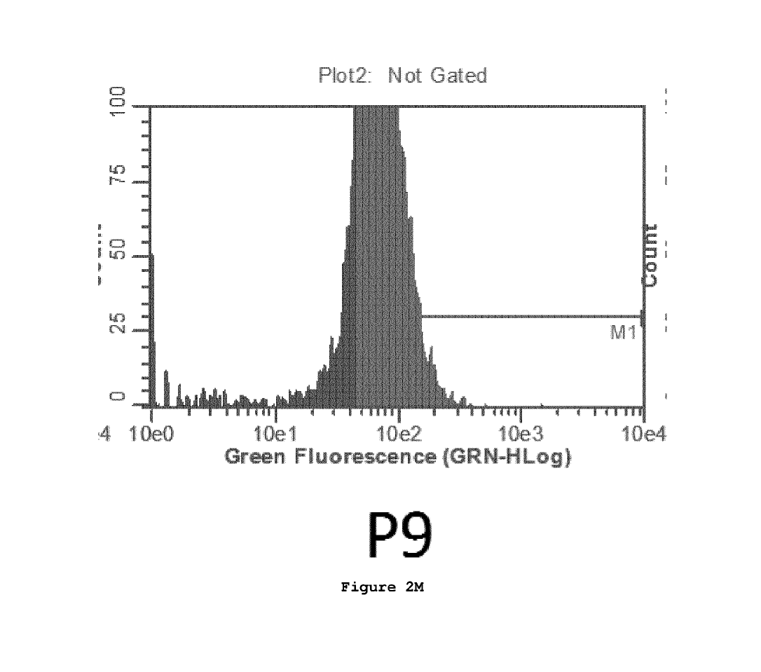

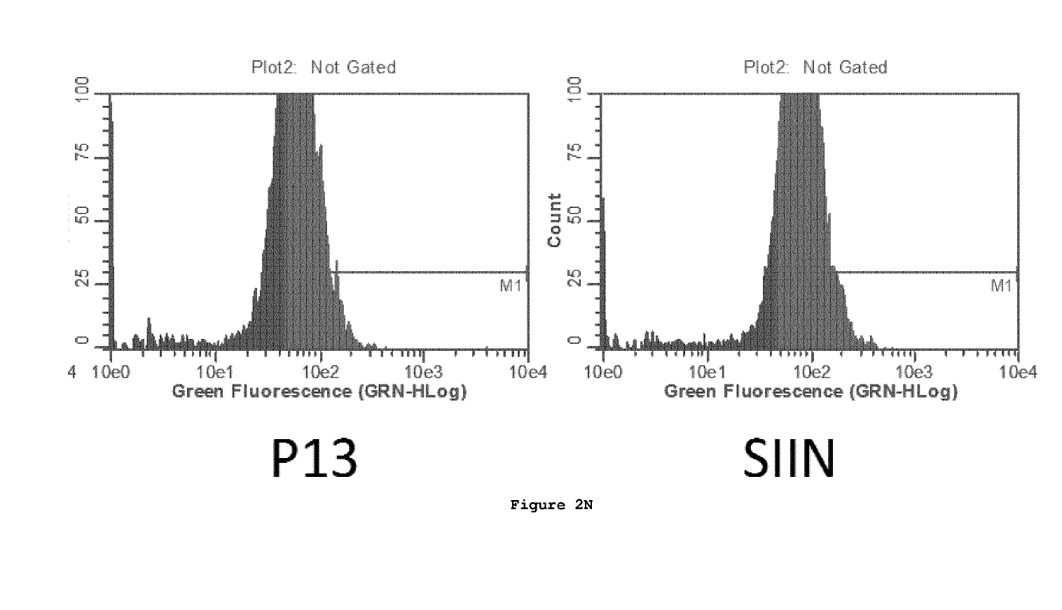

It was important to consider the possible effects of any free peptide present in the nanoparticle samples (FIGS. 2A-B). Therefore, we evaluated how efficiently corresponding free peptide from each preparation is presented. Similar to previous experiments, LKb cells were pulsed for 2 hrs with three concentrations of GNP or free peptide. Presentation was assessed by flow cytometry (FIG. 2C-F) or B3Z assay (FIGS. 2G-H). From these results, we deduced that free peptide is presented well. Lastly, we sought to test whether processing is necessary for presentation of these free peptides. A test requires the pulsing of peptides on ice compared to 37° C. At 37° C., cells can take up peptide and process it within the endosome, however, on ice, peptide can only be loaded on the surface without processing. Indeed, we observed that free peptides with linkers generally need processing while the peptide without the linker can be presented (FIGS. 2I-N) without any processing.

### C. Analysis of Preparations Lacking Free Peptide

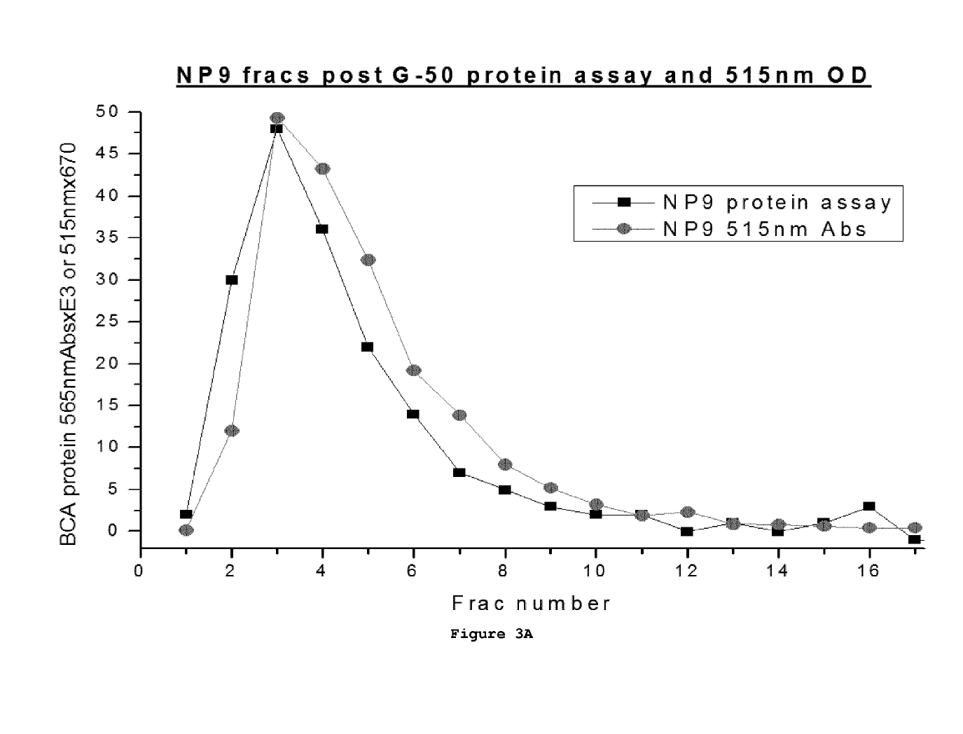

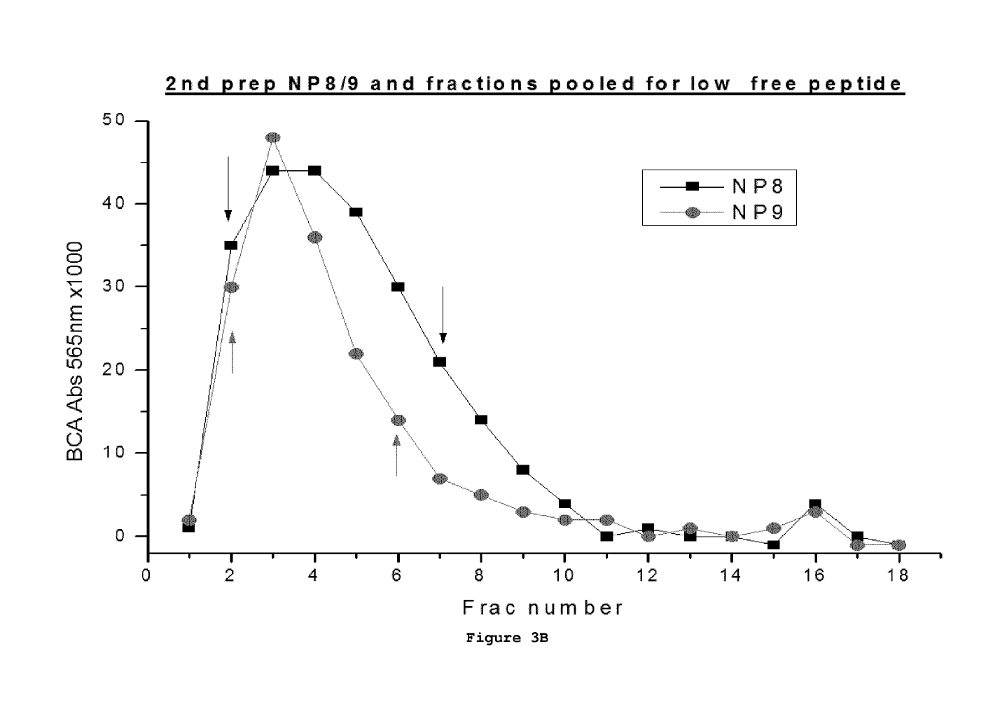

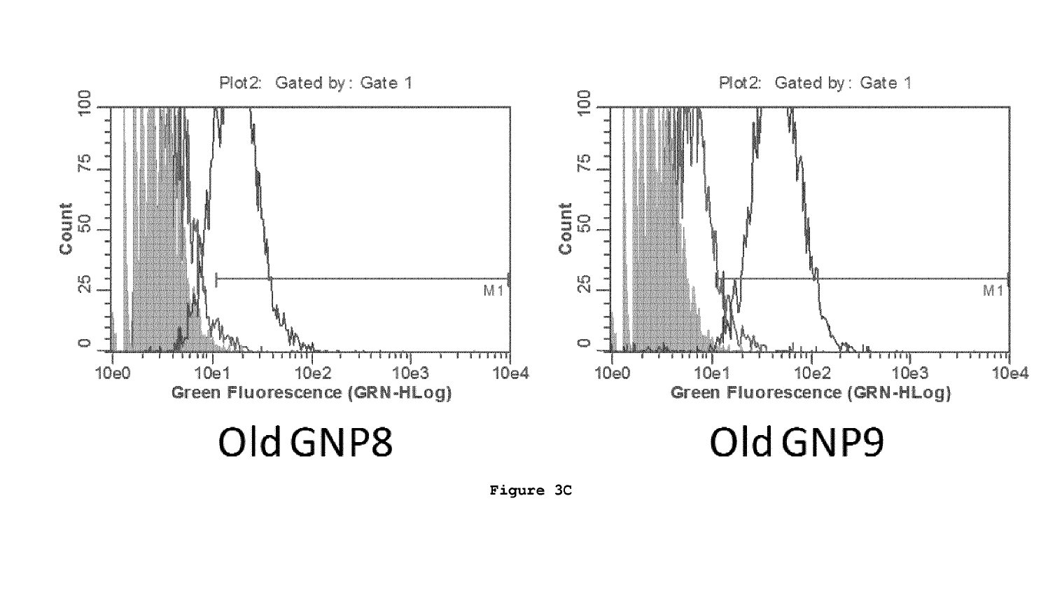

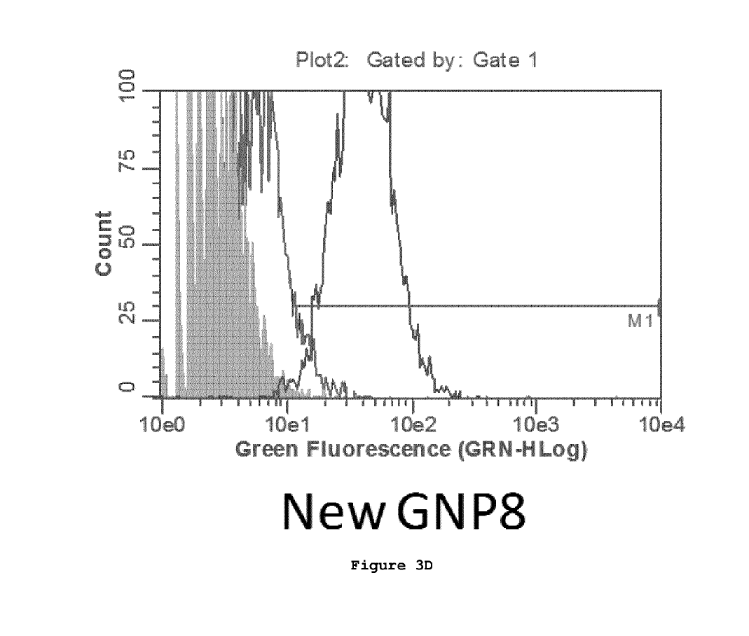

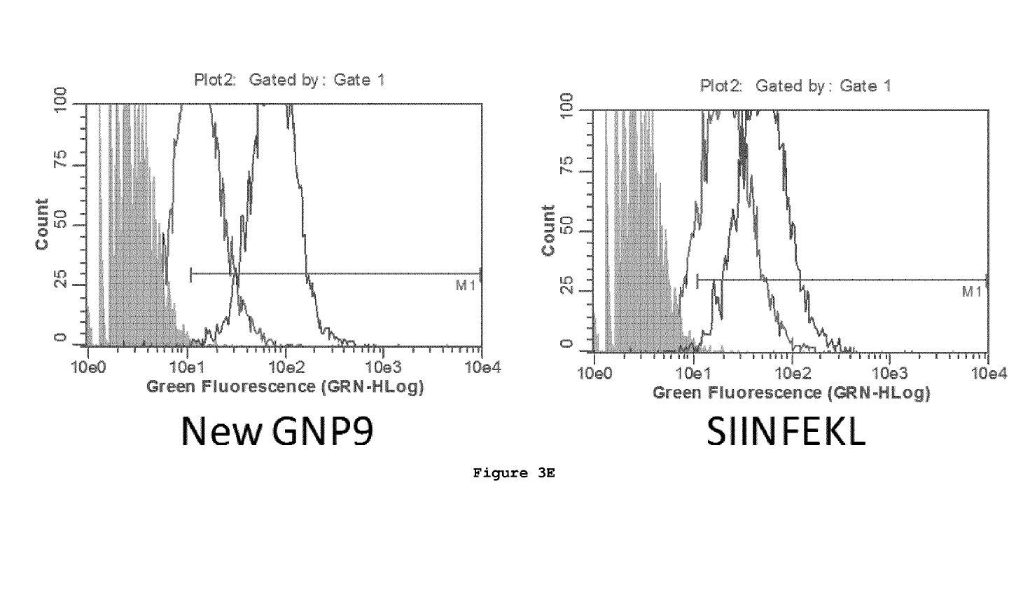

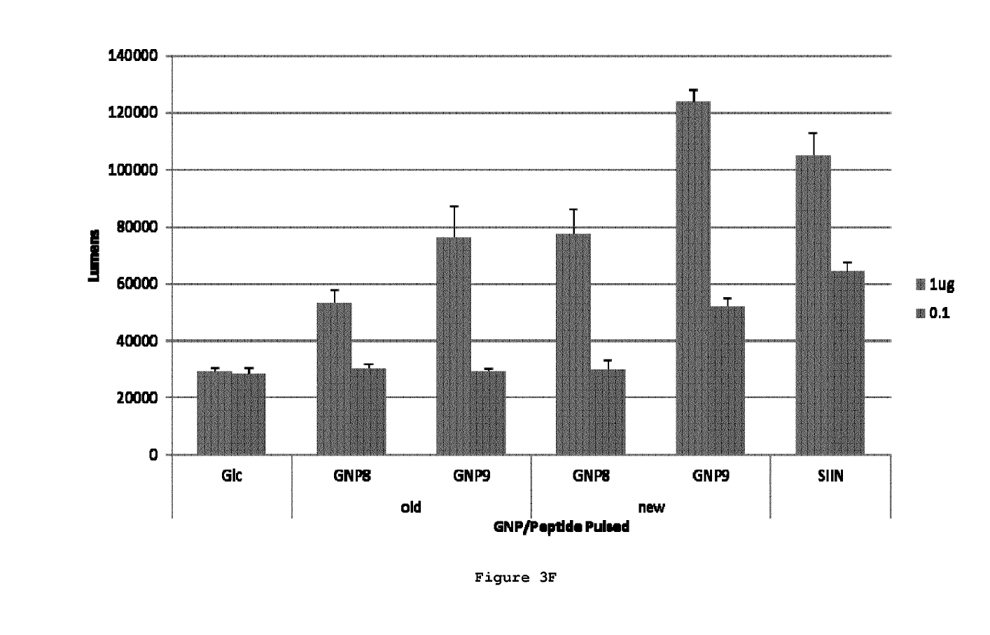

In view of the results demonstrating the possible effect of contaminating free peptides, purified preparations were made. Purification was carried out on the GNPs (8 and 9) using a Sephadex G-50 column removing all free peptide (FIGS. 3A-B). Lastly, above experiments were repeated using previous and newer preparations in the same assay. We extended the two-hour incubation to an overnight period, which has been shown to be sufficient for SIINFEKL presentation (data not shown). Upon flow cytometric analysis, we observed that the samples lacking free peptide work just as well as those containing peptide (FIG. 3C-E). These results were next confirmed using a B3Z assay (FIG. 3F). Therefore, it is clear that GNPs 8 and 9 serve as a viable option for peptide delivery to an antigen presenting cell for presentation of MHCI.

### D. Conclusions

GNPs 8, 9, 12, and 13 are processed and presented very well compared to the others.
This corresponds to the sequences FLAAYSIINFEKL (SEQ ID NO: 89) and AAYSIINFEKL (SEQ ID NO: 90) being the best for in vitro SIINFEKL (SEQ ID NO: 87) presentation (peptide portion of the linker shown underlined).
HS—(CH2)10-(CH2OCH2)7-CONH chemistry is better processed than HS(CH2)2-CONH. However, HS(CH2)2-CONH is also processed very well while being more cost effective.
Contaminating free peptide may give rise to presentation.
Newer preparations, which lack free peptide, are also processed and presented well.
This suggests that GNPs 8 and 9 are efficiently processed for presentation of SIINFEKL (SEQ ID NO: 87).

### Example 4

### Lung Cancer Antigens Delivered Via Nanoparticles Generate Tumour Specific Immune Response In Vivo

### Synthesis of Nanoparticle-Peptide Constructs

Test ligands and their identification numbers are given below (Molecular wt, hydrophilicity score);

(SEQ ID NO: 92)
1. L1 HS(CH2)2-CONH-AAYVLVPVLVMV (1463, −1.3)
(SEQ ID NO: 93)
2. L2 HS(CH2)2-CONH-AAYKIYQWINEL (1601, −0.7)
(SEQ ID NO: 94)
3. L3 HS(CH2)2-CONH-AAYKLGEFAKVLEL (1641, −0.1)
(SEQ ID NO: 95)
4. L4 HS(CH2)2-CONH-AAYGMYGKIAVMEL (1605, −0.6)
(SEQ ID NO: 96)
5. L5 HS(CH2)2-CONH-AAYKLIPFLEKL (1495, −0.3)
(SEQ ID NO: 97)
6. L6 HS(CH2)2-CONH-AAYRLLEVPVML (1463, −0.6)
(SEQ ID NO: 98)
7. P9 HS(CH2)2-CONH-AAYSIINFEKL (1462, −0.4)

Test NPs were synthesized using 10 μmole Gold Chloride (Aldrich 484385), 30 μmole glucose with a thio ethyl linker (GlcC2) and 1.5 μmole of peptide ligand (variable 2.0-2.5 mg).

The following method was used; 1.5 μmole peptide was dissolved in 2 ml methanol, followed by the addition of 30 μmole GlcC2 in 200 μl methanol, and 116 μl of aqueous gold chloride containing 10 μmole Au. The sample was vortexed for 30 sec, shaken for approximately 5 min and then under rapid vortexing for a total of 30 sec 200 μl of 1M NaBH₄ was added, tubes were sealed and then gently shaken for 1.5 h. Samples were bench spun the supernatant removed and the dark pellet resuspended dissolved in 1 ml 95% MeOH/water, vortexed and then recentrifuged, The supernatant was again removed and the NPs dissolved in water and then transferred to a prewashed 10 kDa vivaspin for a total of 4 times 2 ml water washes each of 8 min at SKrpm. NPs were removed from the vivaspins and made up to 600 μl with water, they were then subjected to 15 Krpm bench spin to remove any large aggregates.

Gold content post spin/production is shown below 100% yield would be 2.17 mg (determined by assay);

| NPL | Total mg Au |
|---|---|
| 1 | 1.15 |
| 2 | 1.31 |
| 3 | 1.67 |
| 4 | 1.29 |
| 5 | 1.01 |
| 6 | 1.32 |
| NP 9 | 1.72 |

The solubility of some of the ligands in methanol was poor, especially for 1 and 4, but on addition of the acidic gold chloride clearer solutions were generally obtained, although L1 still had some undissolved peptide material. All NPs had some degree of aggregates that could be spun down, these were removed and account for the overall lower Au yield of between 46.5 and 79.3%. This series of NPs had an extra wash step post production in order to reduce contaminating free peptide.

A BCA assay was used to quantitate peptidic material attached to the NPs, data is shown below all samples/standards are shown as mean of three determinations;

### BCA Data Blank Corrected Standards

| | | | |
|---|---|---|---|
| BSA | 2 | μg | 0.165 |
| BSA | 4 | μg | 0.311 |
| BSA | 8 | μg | 0.566 |
| L2 | 1.92 | μg | 0.209 |
| L3 | 2.14 | μg | 0.159 |
| L4 | 2.40 | μg | 0.300 |
| L5 | 2.14 | μg | 0.151 |

| | BCA | NP | | μg in | Total | mg | % |
|---|---|---|---|---|---|---|---|
| | 565 nm | Blank | Corrected | sample | mg | peptide | peptide |
| NP | OD | corrected* | OD | ** | detected | used | incorporated |
| L1 | 0.079 | 0.038 | 0.041 | 0.50 | 0.30 | 2.2 | 13.6 |
| L2 | 0.208 | 0.062 | 0.146 | 1.77 | 0.80 | 2.4 | 33.3 |
| L3 | 0.153 | 0.060 | 0.093 | 1.13 | 0.75 | 2.5 | 30.0 |
| L4 | 0.176 | 0.036 | 0.140 | 1.70 | 0.67 | 2.4 | 27.9 |
| L5 | 0.072 | 0.026 | 0.046 | 0.56 | 0.39 | 2.2 | 17.7 |
| L6 | 0.087 | 0.034 | 0.053 | 0.64 | 0.38 | 2.2 | 17.2 |
| NP9 | 0.189 | 0.054 | 0.135 | 1.64 | 0.98 | 2.0 | 49.0 |

*This correction is applied as the NPs have some absorbance at 565 nm.
** Samples L1, L6 and NP9 were quantitated using the 2 μg BSA std as these peptides at low levels were insoluble in PBS used in this assay, the remaining 4 NPs used their respective free peptide standards. Assay performed on 1 μl equivalent, 600 μl total prep size.

Table showing material used in subsequent testing, values all shown as mg/ml.

| | | Theoretical | Actual |
|---|---|---|---|
| | Gold level | expected | measured |
| NP | mg/ml | peptide mg/ml | peptide mg/ml |
| L1 | 1.92 | 0.58 | 0.50 |
| L2 | 2.18 | 0.63 | 1.33 |
| L3 | 2.78 | 0.67 | 1.25 |
| L4 | 2.15 | 0.63 | 1.12 |
| L5 | 1.68 | 0.58 | 0.65 |
| L6 | 2.20 | 0.58 | 0.63 |
| NP9 | 2.87 | 0.53 | 1.63 |

### Synthesis of Scaled-Up Batch

Test ligands and their identification numbers are given below (Molecular wt, hydrophilicity score);

(SEQ ID NO: 93)
1. L2 HS(CH2)2-CONH-AAYKIYQWINEL (1601, −0.7)
(SEQ ID NO: 94)
2. L3 HS(CH2)2-CONH-AAYKLGEFAKVLEL (1641, −0.1)
(SEQ ID NO: 95)
3. L4 HS(CH2)2-CONH-AAYGMYGKIAVMEL (1605, −0.6)
4. Glc

Test NPs were synthesized as described above, but at a 3-fold larger scale, using 30 μmole Gold Chloride (Aldrich 484385), 90 μmole glucose with a thio ethyl linker (GlcC2) and 4.5 μmole of peptide ligand (variable 7.2-7.5 mg).

The following method was used; 4.5 μmole peptide was dissolved in 6 ml methanol, followed by the addition of 90 μmole GlcC2 in 600 μl methanol, and 348 μl of aqueous gold chloride containing 30 μmole Au, 50 ml plastic falcons were used as reactant vessels. The sample was vortexed for 30 sec, shaken for approximately 5 min and then under as rapid vortexing as possible for a total of 30 sec 600 μl of 1M NaBH₄ was added, tubes were sealed and then gently shaken for 1.5 h. Samples were bench spun the supernatant removed and the dark pellet resuspended dissolved in 1 ml 95% MeOH/water, vortexed and then recentrifuged, The supernatant was again removed and the NPs dissolved in water and then transferred to a prewashed 10 kDa vivaspin for a total of 4 times 2 ml water washes each of 8 min at SKrpm. NPs were removed from the vivaspins and made up to 1 ml with water, they were then subjected to 15 Krpm bench spin and then transferred to fresh tubes to remove any large aggregates.

Gold content post spin/production by in house assay is shown below 100% yield would be 5.91 mg (determined by assay);

| NPL | Total mg Au |
|---|---|
| 2 | 4.60 |
| 3 | 4.65 |
| 4 | 4.60 |
| Glc | 4.29 |

All NPs had some degree of aggregates that could be spun down, these were removed and account for the overall Au yield of approximately 75%.

This series of NPs had an extra wash step post production in order to reduce contaminating free peptide.

A BCA assay was used to quantitate peptidic material attached to the NPs, data is shown below all samples/standards are shown as mean of three determinations;

### BCA Data Blank Corrected Standards

| | | |
|---|---|---|
| BSA | 2 μg | 0.138 |
| BSA | 4 μg | 0.260 |

| | BCA | NP | | Total | mg | % |
|---|---|---|---|---|---|---|
| | 565 nm | Blank | Corrected | mg/ml | peptide | peptide |
| NP | OD | corrected* | OD | detected | used | incorporated |
| L2 | 0.199 | 0.046 | 0.153 | 4.43 | 7.2 | 33.3 |
| L3 | 0.117 | 0.050 | 0.067 | 1.94 | 7.5 | 30.0 |
| L4 | 0.156 | 0.047 | 0.109 | 3.16 | 7.2 | 27.9 |

*This correction is applied as the NPs have some absorbance at 565 nm.
**Samples were quantitated using the 2 g BSA std, assay performed on 0.5 μl, approx 1 ml total prep size.

### Immunization Methodology

Human HLA transgenic mice were immunized with gold nanoparticles (GNPs) with lung cancer antigen epitopic peptides attached (synthesised as described above). CTLs were assayed against peptide-loaded targets (T2) and lung tumour cells (Lung T1=SCLC; Lung T2=NSCLC; Lung T3=Adenocarcinoma). Controls were anigen unpulsed T2 and N lung=normal lung cells.

The immunization schedule consisted of 3 immunizations (part i.d. and part s.c.), 10 days apart and the spleens were taken out 8 days after the last immunizations before the assay. The GNPs had no adjuvants added, but the free peptides were mixed with montanide adjuvant (incomplete Freund's adjuvant) for immunizations. For in vivo studies, 10 μg/mouse per injection was used. The free peptides were used at the same concentration.

The CTL response was measured by the number of IFN-gamma producing cells per million splenocytes.

Lung Cancer Antigens

| Epitopes | Protein | Function |
|---|---|---|
| L1 - VLV | Discoidin, CUB and | Increased in lung cancers during the |
| | LCCL domain- | process of tumor progression |
| | containing protein 2 | |
| | precursor | |
| L2 - KIY | Cell differentiation | Novel transcriptional cofactor that |
| | protein RCD1 | mediates retinoic acid-induced cell |
| | homolog | differentiation |
| L3 - DQF | ELP2₋ HUMAN | May play a role in chromatin |
| | Elongator | remodeling and is involved in |
| | complex protein 2 | acetylation of histones H3 |
| | (ELP2) | |
| L4 - KLG | Serine/threonine- | Putative human tumor suppressor gene, |
| | protein phosphatase | deregulated in multiple human cancers |
| | 2A 65 kDa | |
| | regulatory | |
| | subunit A beta | |
| | isoform | |
| L5 - GMY | DNA | Mediates ocogene-Induced p16^{INK4a} |
| | damage-binding | activation; Maintains genome |
| | protein 1 (DDB1) | integrity through regulation of Cdt1 |
| L6 - KLI | CTD small | Tumor suppressor gene, implicated in |
| | phosphatase-like | lung cancer and other epithelial |
| | protein 2 | tumors |
| | (CTDSPL2, | |
| | RBSP3) | |
| L7 - KLS | Heat shock 70 kDa | play an important role in hypoxia |
| | protein 5 | tolerance |
| L8 - RLL | ISOC2_HUMAN | Inhibits the expression of p16^{INK4a}, |
| | Isochorismatase | suggesting a role during tumor |
| | domain- | development |
| | containing | |
| | protein 2 | |

### Immunization Results and Conclusions

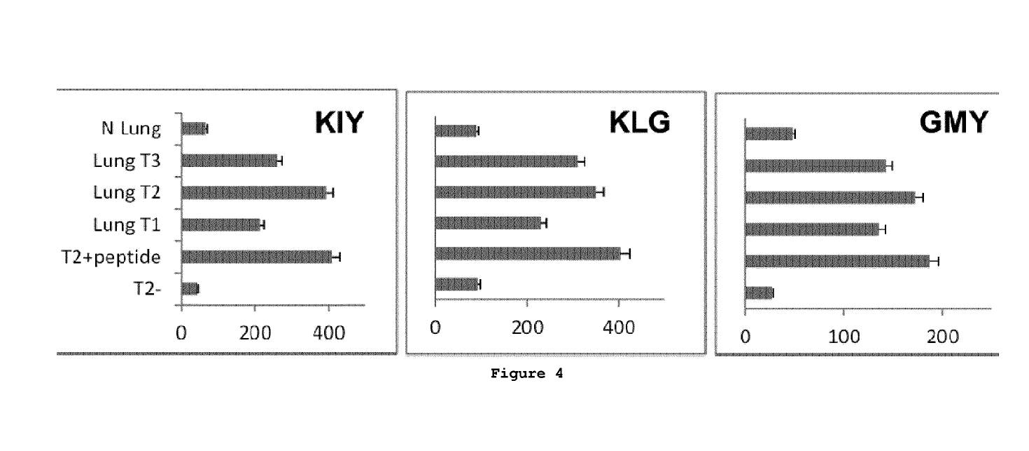

FIG. 4 shows the results of CTL response measured by number of IFN-gamma producing cells per million splenocytes. The lung cancer antigens present on the GNPs were found to generate tumour-specific immune response in vivo.

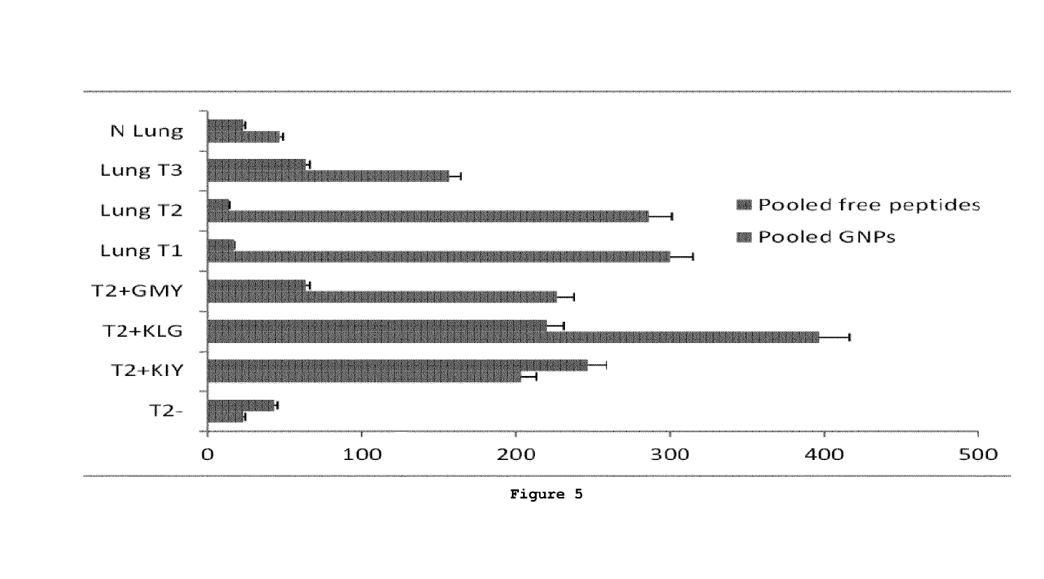

FIG. 5 shows the results of CTL response measured by number of IFN-gamma producing cells per million splenocytes following immunization with pooled GNPs, the pool comprising nanoparticles with three different lung cancer antigens (designated GMY, KLG and KIY).

GMY represents the GNP having the ligand:

HS(CH2)2-CONH-AAYGMYGKIAVMEL (SEQ ID NO: 95)

KLG represents the GNP having the ligand: HS(CH2)2-CONH-AAYKLGEFAKVLEL (SEQ ID NO: 94)

KIY represents the GNP having the ligand:

HS(CH2)2-CONH-AAYKIYQWINEL (SEQ ID NO: 93)

The free peptide control consisted of a pool of the same three epitopic peptides absent the linkers and GNP (“Pooled free peptides”).

As shown in FIG. 5, the tumour specific CTL response was significantly higher in GNP immunized mice compared with the pooled free peptide control. The peptide pulsed target response was found to be comparable. Without wishing to be bound by any theory, it is presently believed that the GNP-bound peptides may stimulate high avidity CTLs in comparison with the free peptides.

The requirements for the high avidity tumour specific T cell response indicate that subdominant epitopes (medium and low MHC binding affinity) are more effective in generating high avidity CTLs. The epitopes tested herein are naturally presented, and are likely to be medium and/or low affinity epitopes. In view of the combination of subdominant epitopes in low concentrations in NPs which are believed to be targeted to APCs, the present results suggest generation of high avidity CTLs.

### Human PBMC In Vitro Assay Results and Conclusions

Human peripheral blood mononuclear cells (PBMCs) were stimulated with GNPs containing a pool of 6 lung cancer antigens (designated VLV, KIY, KLG, GMY, KLI and RLL). These designations correspond to GNPs with the following ligands attached (the underlined portion corresponding to the designation):

(SEQ ID NO: 92)
HS(CH2)2-CONH-AAYVLVPVLVMV
(SEQ ID NO: 93)
HS(CH2)2-CONH-AAYKIYQWINEL
(SEQ ID NO: 94)
HS(CH2)2-CONH-AAYKLGEFAKVLEL
(SEQ ID NO: 95)
HS(CH2)2-CONH-AAYGMYGKIAVMEL
(SEQ ID NO: 96)
HS(CH2)2-CONH-AAYKLIPFLEKL
(SEQ ID NO: 97)
HS(CH2)2-CONH-AAYRLLEVPVML

The GNP-peptide dose used was 10 μg/ml/10 million cells.

Pooled free peptides were used as controls at a dose of 50 μg/ml/10 million cells.

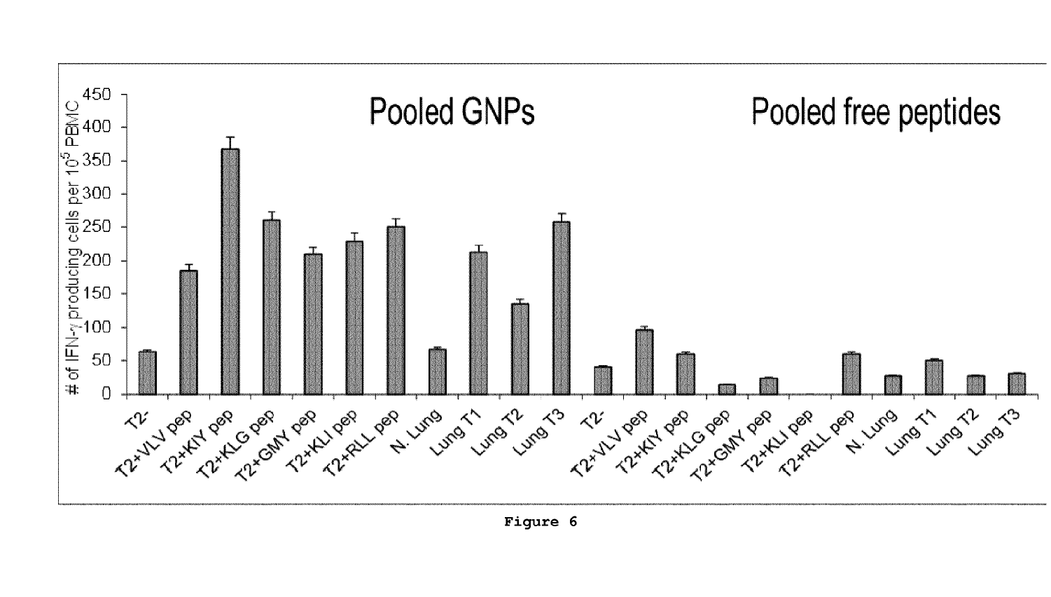

As shown in FIG. 6, tumour specific and Peptide pulsed target CTL response significantly higher in GNP stimulated PBMCs. This shows that lung cancer antigens delivered via GNPs generate a tumour specific immune response in vitro.

### Example 5

### Comparison of Different Nanoparticle Coronas

### Synthesis of NPs with Various Coronas

2.25 μmole of peptide was dissolved in 3 ml methanol (3 individual lung based peptide antigens were tested+a blank), followed by the addition of 45 μmole GlcC2 (glucose having a C₂ linker) in 300 μl methanol, and 100 μl of aqueous gold chloride containing 15 μmole Au. The samples were vortexed for 30 sec, shaken for approximately 5 min and then under rapid vortexing for a total of 30 sec 300 μl of 1M NaBH₄ was added, tubes were sealed and then gently shaken for 1.5 h.

Samples were bench spun the supernatant removed and the dark pellet resuspended in 1 ml 90% MeOH/water, vortexed and then recentrifuged, The supernatant was again removed and another 90% MeOH/water wash was then performed a second time. The final NP pellets were dissolved in water and then transferred to a prewashed 10 kDa vivaspin for a total of 4 times 2 ml water washes each of 8 min at 4 k g. NPs were removed from the vivaspins with water, they were then subjected to 18 k g bench spin to remove any large aggregates. Samples were made up to a final volume of 1 ml in water. Gold content was determined, and the peptide content by difference by C18 HPLC both pre and post CN treatment.

This basic method was followed twice more with the following corona variations;
i. Instead of 45 μmole GlcC2, 33.75 μmole GlcC2 and 11.25 μmole GlcNAcC2 (N-acetylglucosamine having a C₂ linker) were used for all 3 peptides preparations+control.
ii. Instead of 45 μmole GlcC2, 4.5 μmole GlcC2 and 40.5 μmole glutathione were used for all 3 peptide preparations+control, glutathione required a higher water ratio to solubilise (25/75%).

The nanoparticles designated “GlcNAc” below therefore comprise a corona having both glucose-containing and N-acetylglucosamine-containing ligands.

The nanoparticles designated “GSH” below therefore comprise a corona having both glucose-containing and glutathione ligands. Glutathione (GSH) is a wholly natural tripeptide used to regulate cells oxidation status, it is also cost-effective and provides a charged surface which provides high water solubility to the nanoparticles in a manner similar to the high water solubility provided by a corona of glucose-containing ligands. Although the nanoparticles designated “GSH” below also include glucose C₂ ligands, and without wishing to be bound by any theory, the present inventors believe that GSH could completely replace the requirement for C2Glc.

### HLA-A2 Transgenic Mouse Model Studies:

Lung antigens (KIY, KLG, GMY) were tested in NPs with Glc, GlcNAc, GSH corona for activation of CTL in vivo in a HLA-A2 transgenic mouse model.

As above, the designations “KIY”, “KLG” and “GMY” correspond to:

(SEQ ID NO: 93)
HS(CH2)3-CONH-AAYKIYQWINEL
(SEQ ID NO: 94)
HS(CH2)3-CONH-AAYKLGEFAKVLEL
(SEQ ID NO: 95)
HS(CH2)3-CONH-AAYGMYGKIAVMEL,

respectively.

Note that in this example a C₃ (propyl) linker was employed as the non-peptide portion of the ligand, while AAY was employed as the peptide portion of the linker; KIYQWINEL (SEQ ID NO: 29), KLGEFAKVLEL (SEQ ID NO: 33) and GMYGKIAVMEL (SEQ ID NO: 19) being the epitopic peptides, respectively.

Pooled 3 lung peptides (KIY, KLG, GMY) in NPs with Glc, GlcNAc, GSH corona, respectively, or free pooled peptides+montanide were used to immunize mice. The free peptides were used in the absence of the AAY linker portion, i.e. the free peptides were KIYQWINEL (SEQ ID NO: 29), KLGEFAKVLEL (SEQ ID NO: 33) and GMYGKIAVMEL (SEQ ID NO: 19).

After 3 immunizations, the splenocytes were assessed for peptide and lung tumor specific CTLs in an IFN-gamma ELISpot assay and CD107a degranulation markers by flow cytometry.

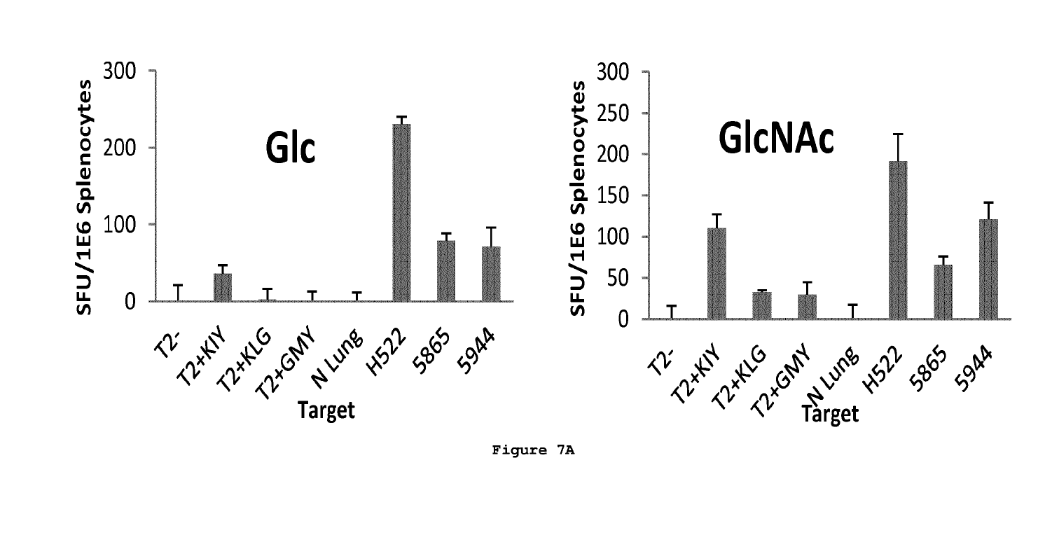

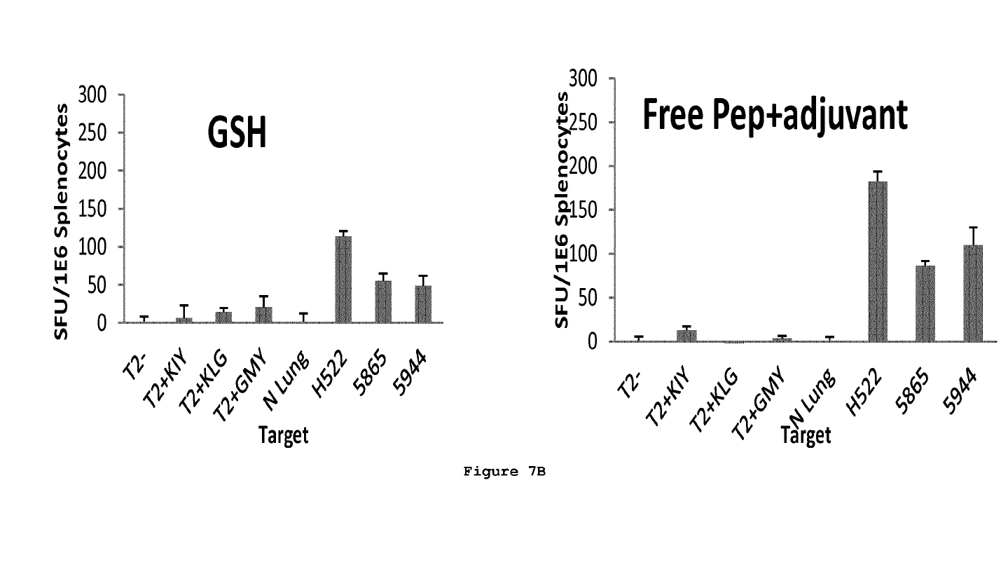

Splenocytes from immunized mice were mixed with various target cells (T2—empty HLA-A2+ cells, N lung—HLA-A2+ normal lung, HLA-A2+ Lung tumor cells—H522, 5865, 5944) to measure IFN-gamma secretion in an ELISpot assay. The data shown in FIGS. 7A and 7B indicate that CTLs were activated by all the three coronas in the NP.

However, peptide specific activation was higher in GlcNAc NPs immunized mice. Importantly, all the three coronas induced equivalent level of tumor specific response. Peptide-loaded NPs without any adjuvant induced equal or higher response as compared to free peptide with montanide-51 adjuvant.

### Antigen Specific Activation Marker (CD8/CD107a) Analysis

In addition to IFN-gamma response, antigen specific CTL degranulation marker CD107a analysis was assessed in the splenocytes in response to peptide loaded T2 cells and well as lung tumor cells.

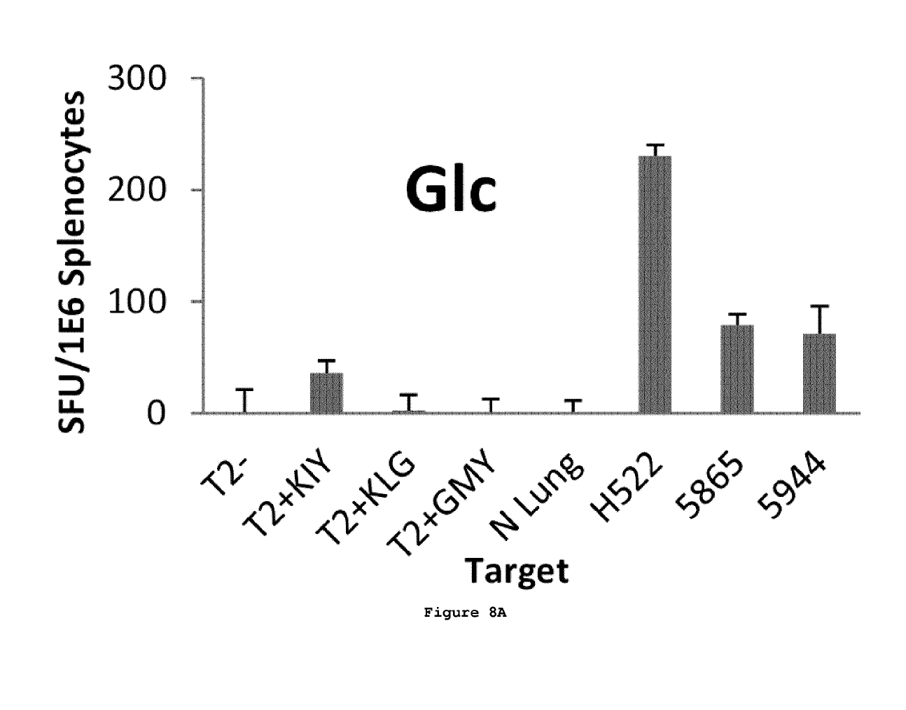

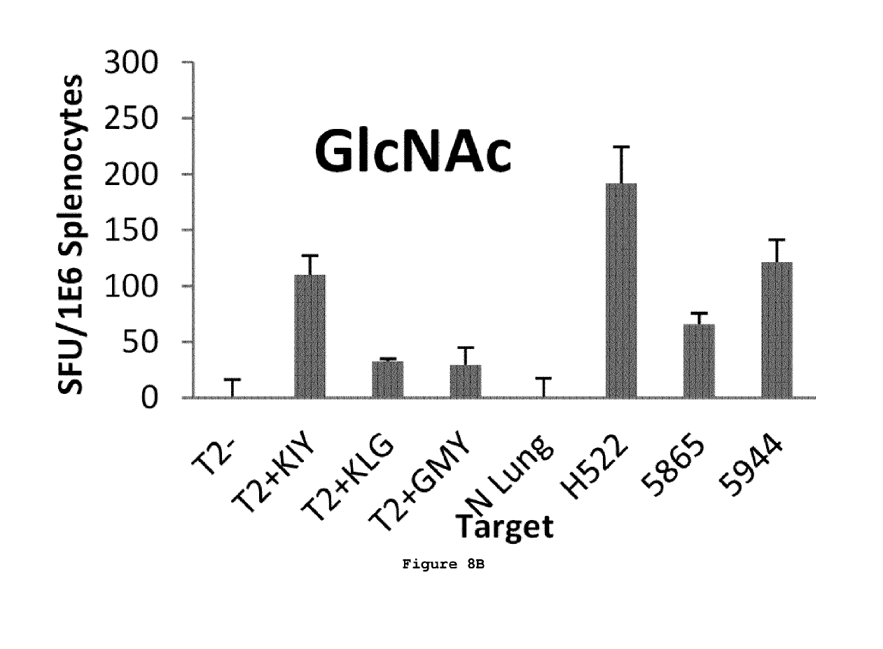

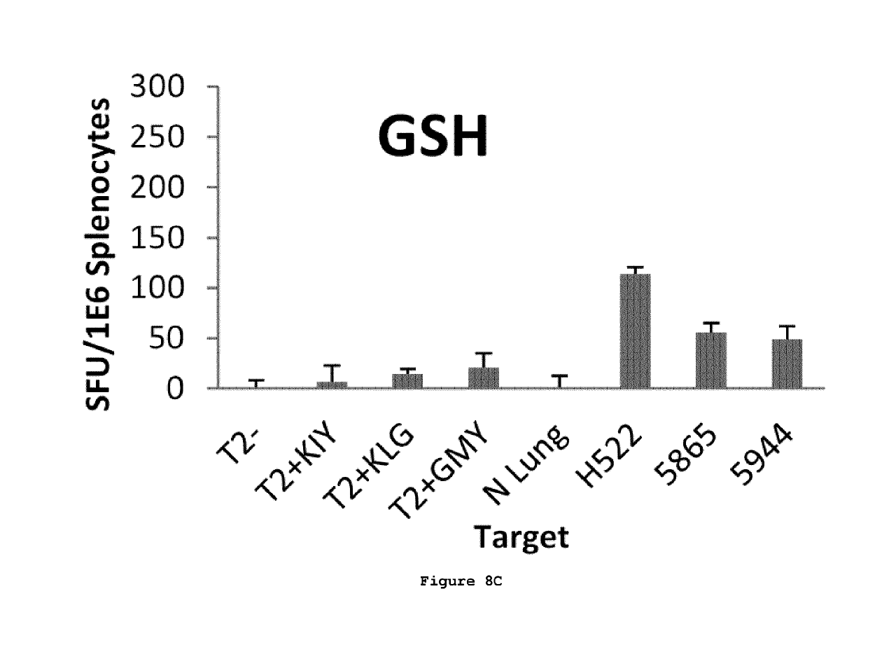

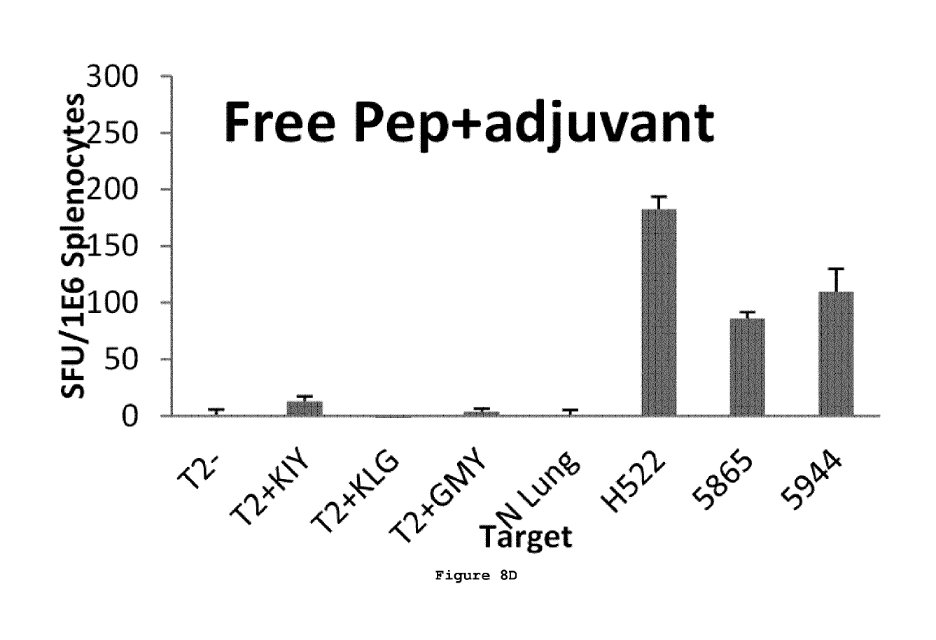

The data shown in FIGS. 8A-8D indicate that NPs with all three coronas without any adjuvant induced active antigen specific CTLs as opposed to the free peptides+montanide-51 adjuvant. More importantly, peptide-loaded NPs induced higher tumor specific CTL activation than the free peptides with adjuvant. Among the coronas, GSH induced higher CTL activation when compared to Glc and GlcNAc.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

4. The vaccine according to claim 1, wherein said peptide portion of said second linker comprises or consists of an amino acid sequence selected from:
(i) AAY;
and
(SEQ ID NO: 91)
(ii) FLAAY.

5. The vaccine according to claim 1, wherein said second linker is selected from the group consisting of:
(i) HS—(CH₂)₂—CONH-AAY;
(ii) HS—(CH₂)₂—CONH-FLAAY;
(iii) HS—(CH₂)₃—CONH-AAY;
(iv) HS—(CH₂)₃—CONH-FLAAY;
(v) HS—(CH₂)₁₀—(CH₂OCH₂)₇—CONH-AAY; and
(vi) HS—(CH₂)₁₀—(CH₂OCH₂)₇—CONH-FLAAY,
wherein said second linker is covalently linked to said core via the thiol group of the non-peptide portion of the linker.

6. The vaccine according to claim 1, wherein said epitopic peptide is linked via its N-terminus to said peptide portion of said second linker.

7. The vaccine according to claim 6, wherein said second ligand is selected from the group consisting of:
(i) HS—(CH₂)₂—CONH-AAYZ₂;
(ii) HS—(CH₂)₂—CONH-FLAAYZ₂;
(iii) HS—(CH₂)₃—CONH-AAYZ₂;
(iv) HS—(CH₂)₃—CONH-FLAAYZ₂;
(v) HS—(CH₂)₁₀—(CH₂OCH₂)₇—CONH-AAYZ₂; and
(vi) HS—(CH₂)₁₀—(CH₂OCH₂)₇—CONH-FLAAYZ₂,
wherein Z₂ represents said epitopic peptide.

1. A vaccine for the prophylactic or therapeutic treatment of a tumour in a mammalian subject, said vaccine comprising a plurality of nanoparticles and a pharmaceutically acceptable carrier, salt or diluent, at least one of said nanoparticles comprising:
(i) a core comprising a metal and/or a semiconductor atom;
(ii) a corona comprising a plurality of ligands covalently linked to the core, wherein at least a first ligand of said plurality comprises a carbohydrate moiety that is covalently linked to the core via a first linker or comprises glutathione, and wherein at least a second ligand of said plurality comprises an epitopic peptide that is covalently linked to the core via a second linker, said second linker comprising:
a peptide portion and a non-peptide portion, wherein said peptide portion comprises the sequence X₁X₂Z₁, wherein:
X₁ is an amino acid selected from A and G;
X₂ is an amino acid selected from A and G; and
Z₁ is an amino acid selected from Y and F,
and wherein said epitopic peptide forms at least a portion of or is derived from a Tumour-Associated Antigen (TAA).

2. The vaccine according to claim 1, wherein said non-peptide portion of the second linker comprises C2-C15 alkyl and/or C2-C15 glycol.

3. The vaccine according to claim 1, wherein said first ligand and/or said second ligand are covalently linked to the core via a sulphur-containing group, an amino-containing group, a phosphate-containing group or an oxygen-containing group.

8. The vaccine according to claim 1, wherein said epitopic peptide binds to a class I Major Histocompatibility Complex (MHC) molecule or is capable of being processed so as to bind to a class I MHC molecule.

9. The vaccine according to claim 8, wherein said epitopic peptide consists of a sequence of 8 to 40 amino acid residues.

10. (canceled)

11. The vaccine according to claim 1, wherein the epitopic peptide is capable of being presented by a class I MHC molecule so as to stimulate a Cytotoxic T Lymphocyte (CTL) response.

12. The vaccine according to claim 1, wherein the TAA is a lung cancer antigen.

13. (canceled)

14. The vaccine according to claim 12, wherein said epitopic peptide comprises or consists of an amino acid sequence selected from SEQ ID NOS: 1 to 86.

15. The vaccine according to claim 14, wherein the epitopic peptide comprises or consists of an amino acid sequence selected from the group consisting of:
(SEQ ID NO: 82)
VLVPVLVMV;
(SEQ ID NO: 29)
KIYQWINEL;
(SEQ ID NO: 33)
KLGEFAKVLEL;
(SEQ ID NO: 19)
GMYGKIAVMEL;
(SEQ ID NO: 34)
KLIPFLEKL;
and
(SEQ ID NO: 67)
RLLEVPVML.

16. The vaccine according to claim 1 wherein:
(i) the carbohydrate moiety of said first ligand comprises a monosaccharide and/or a disaccharide; and/or
(ii) said plurality of ligands comprises at least one glutathione ligand covalently linked to the core of the nanoparticle via the glutathione sulphur atom; and/or
(iii) said plurality of ligands comprises:
(a) glucose;
(b) N-acetylglucosamine;
(c) glutathione;
(d) glucose and N-acetylglucosamine;
(e) glucose and glutathione;
(f) N-acetylglucosamine and glutathionie; or
(g) glucose, N-acetylglucosamine and glutathione.

17. The vaccine according to claim 16(i), wherein said carbohydrate moiety comprises glucose, mannose, fucose and/or N-acetylglucosamine.

18. The vaccine according to claim 1, wherein said first linker comprises C2-C15 alkyl and/or C2-C15 glycol.

19. The vaccine according to claim 1, wherein said first ligand comprises 2′-thioethyl-β-D-glucopyranoside or 2′-thioethyl-α-D-glucopyranoside covalently attached to the core via the thiol sulphur atom.

20.-21. (canceled)

22. The vaccine according to claim 1, wherein the molar ratio of arbohydrate-containing ligands and/or glutathione ligands to epitopic peptide-containing ligands is in the range 5:1 to 100:1.

23. (canceled)

24. The vaccine according to claim 1, wherein the diameter of the core of the nanoparticle is in the range 1 nm to 5 nm.

25. (canceled)

26. The vaccine according to claim 1, wherein the core comprises a metal selected from the group consisting of: Au, Ag, Cu, Pt, Pd, Fe, Co, Gd and Zn, or any combination thereof.

27.-28. (canceled)

29. The vaccine according to claim 1, wherein the core is magnetic.

30. The vaccine according to claim 1, wherein the core further comprises an NMR active atom selected from the group consisting of: Mn²⁺, Gd³⁺, Eu²⁺, Cu²⁺, V²⁺, Co²⁺, Ni²⁺, Fe²⁺, Fe³⁺ and lanthanides³⁺.

31. The vaccine according to claim 1, wherein the core comprises a semiconductor.

32. (canceled)

33. The vaccine according to claim 31, wherein the core is capable of acting as a quantum dot.

34. The vaccine according to claim 1, wherein the at least one nanoparticle comprises at least two epitopic peptide-containing ligands, and wherein the epitopic peptide of each of the at least two epitopic peptide-containing ligands differ.

35. (canceled)

36. A vaccine according to claim 1, wherein the vaccine comprises a first species of said nanoparticle having a first epitopic peptide-containing ligand and a second species of said nanoparticle having a second epitopic peptide-containing ligand, wherein the epitopic peptides of said first and second species differ.

37.-38. (canceled)

39. The vaccine according to claim 1, further comprising at least one adjuvant.

40. (canceled)

41. The vaccine according to claim 39, wherein the adjuvant comprises (S)-(2,3-bis(palmitoyloxy)-(2RS)-propyl)-N-palmitoyl-(R)-Cys-(S)-Ser(S)-Lys₄-OH (“Pam₃Cys”).

42. The vaccine according to claim 1, wherein the vaccine is substantially free of adjuvant or wherein the only adjuvant effect is provided by the nanoparticles.

43.-50. (canceled)

51. A method of prophylactic or therapeutic treatment of a cancer, comprising administering a prophylactically or therapeutically sufficient amount of a vaccine as defined in claim 1 to a mammalian subject in need thereof.

52. A method according to claim 51, wherein said cancer is a lung cancer.

53. (canceled)

54. A method according to claim 51, wherein said vaccine is administered at a site for lymphatic uptake.

55. An in vitro or in vivo method for generating a Cytotoxic T Lymphocyte (CTL) response, comprising:
(i) contacting at least one antigen presenting cell (APC) with a vaccine as defined in claim 1, such that said epitopic peptide is presented on a class I MHC molecule of said APC; and
(ii) contacting said at least one APC of (i) with at least one CTL cell, such that said CTL cell is activated by said APC to generate a CTL response that is specific for said epitopic peptide.

56. An in vitro method according to claim 55, wherein the APC is cultured in the presence of said vaccine, and, simultaneously or sequentially, co-cultured with said CTL cell.

57.-58. (canceled)

59. An in vivo method according to claim 55, wherein said vaccine is delivered by a route of administration selected from the group consisting of:
injection into an organ or tissue of a mammalian subject at, or in the vicinity of, a site of lymphatic uptake;
nasal delivery via a spray or gel;
buccal delivery via a spray or gel or orally dissolvable film;
oral delivery via a dissolvable film
transdermal delivery via a patch incorporating the vaccine; and
inhalation delivery of a composition comprising the vaccine.

60. A method according to claim 55, wherein said vaccine comprises a pool of nanoparticles having different epitoptic peptides.

61. A method according to claim 55, wherein said CTL response comprises production of one or more cytokines.

62. A method according to claim 61, wherein said one or more cytokines comprise interferon gamma (IFN-gamma).

63. A method according to claim 55, wherein said at least one CTL cell exhibits higher avidity for an MHC-peptide complex that comprises said epitopic peptide displayed on a class I MHC molecule, wherein said higher avidity is higher compared with the avidity for said MHC-peptide complex exhibited by a CTL cell activated by an APC that has been contacted with the same epitopic peptide in free peptide form not linked to a nanoparticle.


SEQUENCE LISTING (99 sequences)

SEQ ID NO: 1 - protein, 10 aa, Homo sapiens
AEYDNFFQHL

SEQ ID NO: 2 - protein, 11 aa, Homo sapiens
ALILEPSLYTV

SEQ ID NO: 3 - protein, 10 aa, Homo sapiens
ALLGLDAVQL

SEQ ID NO: 4 - protein, 9 aa, Homo sapiens
DDVPERGLI

SEQ ID NO: 5 - protein, 10 aa, Homo sapiens
DINTDGAVNF

SEQ ID NO: 6 - protein, 11 aa, Homo sapiens
DLDPEAVRGAL

SEQ ID NO: 7 - protein, 9 aa, Homo sapiens
DPFAFIHKI

SEQ ID NO: 8 - protein, 9 aa, Homo sapiens
DQFQKVLSL

SEQ ID NO: 9 - protein, 11 aa, Homo sapiens
DSPVGLAAYIL

SEQ ID NO: 10 - protein, 11 aa, Homo sapiens
DVIVKTPCPVV

SEQ ID NO: 11 - protein, 9 aa, Homo sapiens
EAVRSNLTL

SEQ ID NO: 12 - protein, 10 aa, Homo sapiens
EINTVSIQVK

SEQ ID NO: 13 - protein, 9 aa, Homo sapiens
ELNEILEEI

SEQ ID NO: 14 - protein, 9 aa, Homo sapiens
EMLPNLEIL

SEQ ID NO: 15 - protein, 9 aa, Homo sapiens
EMNIKVPKI

SEQ ID NO: 16 - protein, 12 aa, Homo sapiens
EVEEGESVVLPC

SEQ ID NO: 17 - protein, 9 aa, Homo sapiens
EVVNDLNTL

SEQ ID NO: 18 - protein, 10 aa, Homo sapiens
FLLDGFPRTV

SEQ ID NO: 19 - protein, 11 aa, Homo sapiens
GMYGKIAVMEL

SEQ ID NO: 20 - protein, 8 aa, Homo sapiens
HYVYIINV

SEQ ID NO: 21 - protein, 9 aa, Homo sapiens
ILDVIDDTI

SEQ ID NO: 22 - protein, 11 aa, Homo sapiens
ILTTSVPVYSL

SEQ ID NO: 23 - protein, 9 aa, Homo sapiens
IMPVHLLML

SEQ ID NO: 24 - protein, 8 aa, Homo sapiens
KDNIPMLL

SEQ ID NO: 25 - protein, 11 aa, Homo sapiens
KIFKEEGLLGF

SEQ ID NO: 26 - protein, 9 aa, herpes simplex virus 7
KIKTLLNEL

SEQ ID NO: 27 - protein, 9 aa, Homo sapiens
KILMLQNNQ

SEQ ID NO: 28 - protein, 9 aa, Homo sapiens
KINELNEEL

SEQ ID NO: 29 - protein, 9 aa, Homo sapiens
KIYQWINEL

SEQ ID NO: 30 - protein, 11 aa, Homo sapiens
KLADMLTEITL

SEQ ID NO: 31 - protein, 9 aa, Homo sapiens
KLFDIRPIW

SEQ ID NO: 32 - protein, 9 aa, Homo sapiens
KLFIGGLSF

SEQ ID NO: 33 - protein, 11 aa, Homo sapiens
KLGEFAKVLEL

SEQ ID NO: 34 - protein, 9 aa, Homo sapiens
KLIPFLEKL

SEQ ID NO: 35 - protein, 9 aa, Homo sapiens
KLNEINEKI

SEQ ID NO: 36 - protein, 9 aa, Homo sapiens
KLSLVAAML

SEQ ID NO: 37 - protein, 9 aa, Homo sapiens
KLTSTVMLW

SEQ ID NO: 38 - protein, 9 aa, Homo sapiens
KLYEFVHSF

SEQ ID NO: 39 - protein, 9 aa, Homo sapiens
KLYPWIHQF

SEQ ID NO: 40 - protein, 9 aa, Homo sapiens
KLYQEMFAW

SEQ ID NO: 41 - protein, 9 aa, Homo sapiens
KMFSDEILL

SEQ ID NO: 42 - protein, 9 aa, Homo sapiens
KMFVGGLSW

SEQ ID NO: 43 - protein, 9 aa, Homo sapiens
KSLLFVNTL

SEQ ID NO: 44 - protein, 9 aa, Homo sapiens
LTIKSIITL

SEQ ID NO: 45 - protein, 10 aa, Homo sapiens
LTLLNPRMIV

SEQ ID NO: 46 - protein, 10 aa, Homo sapiens
LVEELSLQEA

SEQ ID NO: 47 - protein, 8 aa, Homo sapiens
LVFQTCDI

SEQ ID NO: 48 - protein, 10 aa, Homo sapiens
LVMSNQKEVL

SEQ ID NO: 49 - protein, 10 aa, Homo sapiens
LVSIVVAVPL

SEQ ID NO: 50 - protein, 9 aa, Homo sapiens
LWPDIGVPI

SEQ ID NO: 51 - protein, 8 aa, Homo sapiens
MDRVLLHV

SEQ ID NO: 52 - protein, 11 aa, Homo sapiens
MEAAMKGLVQE

SEQ ID NO: 53 - protein, 8 aa, Homo sapiens
MGFKPLVL

SEQ ID NO: 54 - protein, 9 aa, Homo sapiens
MLDLHCDKI

SEQ ID NO: 55 - protein, 10 aa, Homo sapiens
MTYDGNNPVT

SEQ ID NO: 56 - protein, 10 aa, Homo sapiens
MVDNPLNQLS

SEQ ID NO: 57 - protein, 8 aa, Homo sapiens
NKDNDKLI

SEQ ID NO: 58 - protein, 11 aa, Homo sapiens
NKEVEEERIAG

SEQ ID NO: 59 - protein, 11 aa, Homo sapiens
QALAQKGLYVT

SEQ ID NO: 60 - protein, 11 aa, Human papillomavirus type 23
QINAMNSDILE

SEQ ID NO: 61 - protein, 9 aa, Homo sapiens
QINEQVEKL

SEQ ID NO: 62 - protein, 9 aa, Homo sapiens
QLDELNQKL

SEQ ID NO: 63 - protein, 12 aa, Homo sapiens
QVHKRGVVEYEA

SEQ ID NO: 64 - protein, 9 aa, Homo sapiens
RIIKWLYSI

SEQ ID NO: 65 - protein, 10 aa, Homo sapiens
RINTVSLKEA

SEQ ID NO: 66 - protein, 11 aa, Homo sapiens
RLFPLSGHLLL

SEQ ID NO: 67 - protein, 9 aa, Homo sapiens
RLLEVPVML

SEQ ID NO: 68 - protein, 10 aa, Homo sapiens
SIMSIDKAVT

SEQ ID NO: 69 - protein, 8 aa, Homo sapiens
SLDRYIKI

SEQ ID NO: 70 - protein, 10 aa, Homo sapiens
SLFAGGMLRV

SEQ ID NO: 71 - protein, 8 aa, Homo sapiens
SLMQCWQL

SEQ ID NO: 72 - protein, 10 aa, Homo sapiens
SLVAKDNGSL

SEQ ID NO: 73 - protein, 10 aa, Homo sapiens
SLVLRKLDHL

SEQ ID NO: 74 - protein, 11 aa, Homo sapiens
SPALALPNLAN

SEQ ID NO: 75 - protein, 12 aa, Homo sapiens
SSSGLISSLNEI

SEQ ID NO: 76 - protein, 8 aa, Homo sapiens
TCFNLLDL

SEQ ID NO: 77 - protein, 11 aa, Homo sapiens
TLTVGTNGGLK

SEQ ID NO: 78 - protein, 11 aa, Homo sapiens
TLVQLPNGQTV

SEQ ID NO: 79 - protein, 10 aa, Homo sapiens
TVFVLDDGTV

SEQ ID NO: 80 - protein, 8 aa, Homo sapiens
TVMMGKKL

SEQ ID NO: 81 - protein, 10 aa, Homo sapiens
VLGAVMIVMV

SEQ ID NO: 82 - protein, 9 aa, Homo sapiens
VLVPVLVMV

SEQ ID NO: 83 - protein, 9 aa, Homo sapiens
VMLPDNQSL

SEQ ID NO: 84 - protein, 12 aa, Homo sapiens
YINQSKAIDYEA

SEQ ID NO: 85 - protein, 9 aa, Homo sapiens
YLLRVVLKT

SEQ ID NO: 86 - protein, 11 aa, Homo sapiens
YVEFTRSLFVN

SEQ ID NO: 87 - protein, 8 aa, Gallus gallus
SIINFEKL

SEQ ID NO: 88 - protein, 10 aa, Artificial sequence
  Synthetic sequence Test ligand
FLSIINFEKL

SEQ ID NO: 89 - protein, 13 aa, Artificial sequence
  Synthetic sequence Test ligand
FLAAYSIINFEKL

SEQ ID NO: 90 - protein, 11 aa, Artificial sequence
  Synthetic sequence Test ligand
AAYSIINFEKL

SEQ ID NO: 91 - protein, 5 aa, Artificial sequence
  Synthetic sequence Peptide portion of linker
FLAAY

SEQ ID NO: 92 - protein, 12 aa, Artificial sequence
  Synthetic sequence Test ligand
AAYVLVPVLVMV

SEQ ID NO: 93 - protein, 12 aa, Artificial sequence
  Synthetic sequence Test ligand
AAYKIYQWINEL

SEQ ID NO: 94 - protein, 14 aa, Artificial sequence
  Synthetic sequence Test ligand
AAYKLGEFAKVLEL

SEQ ID NO: 95 - protein, 14 aa, Artificial sequence
  Synthetic sequence Test ligand
AAYGMYGKIAVMEL

SEQ ID NO: 96 - protein, 12 aa, Artificial sequence
  Synthetic sequence Test ligand
AAYKLIPFLEKL

SEQ ID NO: 97 - protein, 12 aa, Artificial sequence
  Synthetic sequence Test ligand
AAYRLLEVPVML

SEQ ID NO: 98 - protein, 11 aa, Artificial sequence
  Synthetic sequence Test ligand
AAYSIINFEKL

SEQ ID NO: 99 - protein, 4 aa, Gallus gallus
SIIN